(19) United States
(12) Patent Application Publication
(10) Pub. No.: US 2022/0409739 A1
(43) Pub. Date: Dec. 29, 2022

(54) AN EXTRACELLULAR VESICLE

(71) Applicants: Oxford University Innovation Limited, Oxford (Botley) (GB); Cambridge Enterprise Limited, Cambridge (GB)

(72) Inventors: Ghulam Hassan DAR, Oxford (Oxfordshire) (GB); Matthew WOOD, Oxford (Oxfordshire) (GB); Roger A. BAKER, Cambridge (Cambridgeshire) (GB); Wei-Li KUAN, Cambridge (Cambridgeshire) (GB)

(21) Appl. No.: 17/772,526

(22) PCT Filed: Oct. 30, 2020
(86) PCT No.: PCT/GB2020/052758

§ 371 Date: Apr. 28, 2022

(30) Foreign Application Priority Data
Oct. 31, 2019 (GB) 1915855.9

(51) Int. Cl. A61K 47/69; A61K 47/65; C12N 9/02

(52) U.S. Cl. CPC A61K 47/6901; A61K 47/65; C12N 9/0008; C12Y 102/01012

(57) ABSTRACT

The present invention relates to compositions for the delivery of molecules such as a peptide, a nucleic acid and/or a small molecule drug. In particular, the present invention relates to an extracellular vesicle (EV) loaded with a peptide, a nucleic acid and/or a small molecule drug, along with methods of producing said EV.

### FIELD OF THE INVENTION

The present invention relates to compositions for the delivery of molecules such as a peptide, a nucleic acid and/or a small molecule drug. In particular, the present invention relates to an extracellular vesicle (EV) loaded with a peptide, a nucleic acid and/or a small molecule drug, along with methods of producing said EV.

### BACKGROUND TO THE INVENTION

Extracellular vesicles (EV) are natural lipid vesicle nanoparticles secreted by cells for intercellular communication. They also have an important role in pathophysiology. As cells can naturally take up EVs from their surroundings, EVs have great potential for delivery of molecules of interest. However, at present, there is no effective method available to load EVs with a specific molecule of interest, such as a therapeutic nucleic acid. Methods reported earlier, such as electroporation, chemical transfection and lipid mediated conjugation lack efficiency and reproducibility. Therefore, one of the limiting factors in EV therapeutics is the lack of efficient and reproducible method to load a specific molecule of interest into EVs.

It has been reported that a phosphatidylserine-(PS) binding domain in the glyceraldehyde-3-phosphate dehydrogenase (GAPDH) protein can mediate binding of GAPDH to the nuclear envelop via PS lipids in the nuclear membrane (Kaneda et al). It has also been reported that cells uptake iron from the media by secreting EVs containing GAPDH protein on their outer surface (Malhotra et al). In addition, the GAPDH protein has also been found to participate in formation of nuclear membrane after cell division by fusing small nuclear vesicles (Han et al; Nakagawa et al). Further, it has been reported that chloroquine may enhance release of particles from late-endosomes (Lonn et al).

However, PS-binding proteins have not been employed to load EVs in general with a molecule of interest. Further, it has not been shown that high levels of PS-binding proteins can be bound to the outer surface of the EV, let alone how to produce an EV having a high levels of PS-binding proteins bound to their outer surface. In addition, proteins that may bind PS, such as GAPDH, have been shown to cause fusion of synthetic vesicles containing phosphatidylserine and plasmenylethanolamine lipids (Glaser and Gross, 1995; Tisdale, 2001). Such fusogenic properties would lead to clumping (i.e. aggregation) of EVs, which may be undesirable in the context of an EV delivery system and/or a therapeutic delivery system.

The inherent limitations of current strategies for loading EVs have generally limited industrial applications of EVs, including potential therapeutic applications, such as delivery of specific molecules of interest. With the increasing number of diseases shown to possess a genetic component, including neurological disease, obesity and heart disease, there is tremendous potential for the modification of susceptibility genes for preemptive genetic solutions, but only if limitations are overcome. There is also potential to use loaded EVs as an immunogen or as a reagent in a research and development setting.

Hence, it is imperative to develop technologies for the effective loading of EVs with a molecule of interest. One of the solutions may lie in the efficient loading an EV with a molecule of interest.

### SUMMARY OF THE INVENTION

The inventors have successfully provided an EV having a PS-binding protein bound to the outer surface of the EV. An example of a protein that may bind to the surface of an EV and/or to a PS lipid is GAPDH. EVs isolated from different cellular sources are able to bind GAPDH via free GAPDH binding sites on the surface of EVs. Further, it was found that GAPDH comprised motifs that mediate binding of GAPDH to the surface of EVs. An example of such a motif is the G58 motif, which is locate between the 70 to 90 amino acid regions of GAPDH.

The inventors were able to ensure that compositions comprising said EVs were substantially devoid of vesicle aggregates and/or had a significant number of lipid-binding proteins such as PS-binding proteins bound to the outer surface of each EV. The lipid-binding protein and/or GAPDH may be linked to a second molecule, such as a second protein and/or peptide. The second protein and/or peptide may be a cargo-binding protein and/or peptide, such as a nucleic acid-binding protein and/or peptide. Alternatively, or in addition, the lipid-binding protein and/or GAPDH may be linked to a small molecule drug. Thus, the present invention relates to EVs having a lipid-binding protein bound to the outer surface of the EV. Thus, the present invention also relates to EVs having a GAPDH molecule bound to the outer surface of the EV. The EVs may be loaded with a cargo, such as a nucleic acid and/or a small molecule drug. The cargo be bound directly to the lipid-binding protein and/or bound to the second molecule that is linked to the lipid-binding protein. The cargo be bound directly to the GAPDH and/or bound to the second molecule that is linked to GAPDH.

To determine, whether exogenous binding of GAPDH to EV surface may be mediated by the PS-binding domain of GAPDH, the inventors cloned the PS-binding peptide of GAPDH (designated as G58) into a vector, such as pET28, and expressed G58 in E. coli and isolated the G58 peptide. Incubation of G58 peptide with EVs resulted in efficient binding of the peptide to the surface of an EV. Moreover, the inventors did not observe any change in size of EVs modified with G58 peptide, in contrast to the use of full-length GAPDH, thus overcoming the role of GAPDH tetramerization in aggregation and/or fusion of EVs. In other words, the inventors overcame the aggregation of EVs.

Due to the surprising abundant binding of GAPDH protein to the EV surface, the inventors were able to utilise their new methodology to load, in a single step, nucleic acid externally onto the surface of EVs after purification of EVs from different sources. The interaction identified by the inventors also allowed the successful purification of EVs. The method allows for the use of EVs to deliver molecules of interest, such as therapeutic nucleic acid, into targeted tissues, for example to silence disease-causing genes. By way of example, the inventors utilised G58 peptide to load siRNA onto the surface of an EV. The inventors also fused different types of nucleic acid binding peptides to G58 peptide and assessed their binding to EV surface. All G58 fusion proteins showed efficient binding to the surface of an EV and enabled the remarkable loading of siRNA into EVs. Treatment of cells, such as N2a cells, with siRNA loaded EVs resulted in efficient uptake and silencing of genes in the cells. The silencing efficiency increased when cells were treated with endosomolytic molecules such as chloroquine that enable release of the siRNA from late endosomes, via a phenomenon termed the “proton sponge phenomenon”. Other endosomolytic molecules could also be used, such as endosomolytic variants of chloroquine, such as hydroxychloroquine.

Thus, the present invention also relates to methods for loading an EV with a molecule of interest such as a nucleic acid and/or a small molecule drug, as well as to uses of loaded EVs such as for the therapeutic delivery of the molecule of interest.

In accordance with a first aspect of the present disclosure, there is provided a composition comprising an extracellular vesicle (EV), further comprising a phosphatidylserine-binding protein and/or peptide bound to the outer surface of the EV by means of an interaction between the phosphatidylserine-binding protein and/or peptide, and a lipid and/or an EV protein on the outer surface of the EV. The lipid may be the phospholipid phosphatidylserine. The composition of the first aspect of the present disclosure may be combined with at least one pharmaceutically acceptable excipient, for use in a method of therapy in a subject.

In accordance with a second aspect of the present disclosure, there is provided a method of producing the composition according to the first aspect, comprising:
(a) providing an EV expressing phosphatidylserine on the outer surface of the EV; and
(b) providing a phosphatidylserine-binding protein and/or peptide to the EV and allowing the phosphatidylserine-binding protein and/or peptide to bind to the phosphatidylserine,

thereby producing an EV composition comprising a phosphatidylserine-binding protein and/or peptide bound to the outer surface of the EV by means of an interaction between the phosphatidylserine-binding protein and/or peptide and phosphatidylserine on the outer surface of the EV.

In accordance with a third aspect the present disclosure, there is provided a protein and/or peptide and or/peptide comprising:
(a) a polypeptide sequence having at least 80%, at least 90%, at least 95% or at least 100% sequence identity to SEQ ID NO: 1, optionally comprising 1-10 additional amino acids at the 5′ and/or 3′ end;
(b) a polypeptide having at least 80%, at least 90%, at least 95% or at least 100% sequence identity to SEQ ID NO: 2; and/or
(c) at least 10, at least 20 or at least 30 contiguous amino acid residues from the polypeptide sequence of SEQ ID NOs: 1 or 2.

In accordance with a fourth aspect of the present disclosure, there is provided a composition according to the first aspect, or a protein and/or peptide according to the third aspect, for purifying an EV.

In accordance with a fifth aspect of the present disclosure, there is provided an in vitro or ex vivo use of a composition according to the first aspect, or a protein and/or peptide according to the third aspect, as a research tool, a diagnostic tool, an imaging tool, biological reference material, an experimental control and/or an experimental standard.

### DESCRIPTION OF THE FIGURES

FIG. 1 Surface binding of GAPDH leads to aggregation of EVs

(a) Western blot showing exogenous binding of GAPDH to HEK293T EVs. Increasing concentrations of histidine- (His6-) and Flag-tagged GAPDH (lane 3, 4 and 5) were incubated with a fixed number of EVs (see details in methods). Endogenous GAPDH (Endo. GAPDH) present naturally on EV surface is shown in the top blot along with the exogenous GAPDH (Exo. GAPDH). Alix and CD81 are EV protein markers used as a positive control. Calnexin (bottom blot) was used to demonstrate the purity of EV samples. In this blot lane 2, 3 and 4 represent EVs, and lane 5 represents cell lysate. Representative blots (n>3). (b) UV-absorbance spectrum of EVs after passing through gel-filtration column. Increase in the absorbance of EVs+GAPDH peak indicated binding of GAPDH protein. Purified EVs were used for incubation with either GAPDH (upper chromatogram) or BSA protein (lower chromatogram). The first peak (at ˜10 ml elution) represents EVs and the second peak (around 20 ml elution) represents unbound proteins. Representative graphs (n>3) (c) NTA profile showing the size distribution of purified HEK293T EVs after incubation with either GAPDH or BSA proteins respectively. Binding of GAPDH to the EVs, shifted their size. Inset is the scatter plot representing size of EVs (red; EVs+GAPDH, black; EVs+BSA). Data shown as mean±s.d, n=9***p<0.0001 when compared to EVs+BSA (two tailed t-test) (d) Electron microscopy images of EVs incubated with either BSA or GAPDH protein. The EVs from HEK293T cells were passed through a gel-filtration column to remove excess of unbound GAPDH. Uranyl oxalate, pH 7 was used to stain the EVs. Images were collected at 120 kVon FEI Tecnai 12 TEM with Gatan one view CMOS Camera. Representative images (n=2). (Whole images are in supporting document FIG. 3a). (e) Photographic images (taken with Canon EOS 200D) of tubes showing the formation of thread like structures from MSCs and HeLa EVs after incubation with GAPDH for 2 h at 4° C. Representative images (n=2). (Whole images are in supporting data, FIG. 3b). (f) Western blot showing binding of G58 peptide to HEK293T (designated as 293T) and MSCs EVs. Second domain of TRBP protein was attached to G58 peptide for detection by TRBP2 antibody. Representative blots (n>4). (g) NTA profile showing the size distribution of HEK293T EVs after binding to the G58T protein. Inset is the scatter plot representing size of EVs after incubating with either BSA (black) or G58T protein (red). Data shown as mean±s.d, n=9 (ns=non-significant, two tailed t-test). Binding of G58T protein to EVs did not significantly alter the size of EVs. Representative graph (n>3).

FIG. 2 GAPDH regulates EV (exosome) biogenesis and clustering in Drosophila secondary cells

Schematic at the top of FIG. 2 shows a male fruit fly and its accessory gland (AG) containing main cells and secondary cells (SCs), which are only found at the distal tip of the gland. Exosomes can be visualised at the AG lumen as fluorescent puncta. A schematic of a secondary cell expressing a GFP-tagged form of Breathless (Btl-GFP; green) is also shown. The Rab11 compartments, which contain intraluminal vesicles (ILVs; green) and dense-core granules (DCGs; dark grey), and the late endosomes and lysosomes (magenta) are marked. Panels A-D show basal wide-field fluorescence and differential interference contrast (DIC) views of living secondary cells (SCs) expressing a GFP-tagged form of Breathless (Btl-GFP; green). SC outline approximated by dashed white circles, and acidic compartments are marked by LysoTracker Red (magenta). A single non-acidic compartment containing a dense-core granule (DCG) and intraluminal vesicles (ILVs) is boxed and magnified in ‘Zoom’. DCG compartment outline is approximated by white circles. Confocal transverse images of fixed accessory gland (AG) lumens are shown on the right. (A) SC from 6-day-old male expressing Btl-GFP, but no RNAi construct (control). Btl-GFP-positive ILV membranes are apparent inside compartments (arrowheads; Zoom), surrounding the DCG (asterisk) and connecting it to the limiting membrane of the compartment, and as puncta in AG lumen (arrowheads). (B) SC also expressing human GAPDH protein, hsGAPDH. Btl-GFP-positive ILVs (Zoom) and puncta in the AG lumen are increased with luminal puncta frequently clustered. The DCG is properly formed. (C) SC expressing RNAi construct targeting Drosophila GAPDH1. Btl-GFP-positive ILVs (Zoom) are present inside compartments, and number of puncta in the lumen is not significantly reduced. DCG formation is not affected (Zoom). (D) SC expressing RNAi construct targeting Drosophila GAPDH2. The majority of Btl-GFP-positive ILVs are attached to the limiting membrane of DCG compartments (Zoom), with the number of internal puncta significantly reduced. DCGs are severely disrupted (“split DCG”; asterices), with small DCGs attached to the limiting membrane (Zoom). (E) Bar chart showing the percentage of cells containing compartments with “split DCGs”. —to come (F) Bar chart showing Btl-GFP fluorescent puncta number in the lumen of AGs with gapdh1 and gapdh2 knockdown compared to control SCs. All data are from six-day-old male flies shifted to 29° C. at eclosion to induce expression of transgenes. Genotypes are: w; P[w+, tub-GAL80ts]/+; dsx-GAL4, P[w+, UAS-btl-GFP]/+ with no additional overexpression or knockdown construct (A), UAS-hsGAPDH (v79196; B), UAS-gapdh1-RNAi (BL #36842; C) or UAS-gapdh2-RNAi (BL #26302; D). Scale bars in A-D (5 μm); in AG lumen (20 μm).

FIG. 3 G58 peptide promotes EV-mediated siRNA delivery to the brain

(a) Confocal microscopy image of Neuro-2a cells after 4 h of incubation with siRNA-loaded G58T EVs. Nuclei of cells were stained with Hoechst 3342. EV surface proteins were labelled with Alexa fluor-633 (red colour) and siRNA was labelled with Cy-3 dye (green). Inset is the magnified image of the marked region showing colocalization of EVs with siRNA (yellow spots). Scale bar, 10 μm. Representative image (n>3). The whole image is in supporting document FIG. 3. (b) Silencing of GAPDH expression in N2a cells by EVs engineered with G58T, G58T(tat)2 and G58TF proteins, respectively. 30 nM of GAPDH siRNA was loaded into EVs and added to cells. Top histogram represents GAPDH mRNA level determined after 48 h of treatment, using probe-based qRT-PCR. Data were normalized with 18S rRNA. GAPDH mRNA levels in saline-treated cells, defined as 1, were used to calculate relative mRNA expression level in treated cells. Mock; G58TF-bound EVs alone, Neg. Control; G58TF EVs+negative siRNA. Results shown as mean±s.d. **p=0.0006 and ***p<0.0001 when compared to control (One way ANOVA; n=3). Bottom western blot shows GAPDH protein level in N2a cells after 72 h of treatment. A single label is used for histogram and western blot images. Representative blot (n=3) (c) Western blot showing effect of chloroquine (CQ) on silencing of GAPDH protein by G58T and G58TF EVs in N2a cells. (Mock: G58TF EVs alone, Neg. Control: G58TF EVs+negative siRNA, RNai max: lipofectamine RNAiMAX reagent (Invitrogen). 30 μM chloroquine was added to cells along with the EVs. Representative blot (n=2) (d) In vivo fluorescence images of C57BL/6 mice brain showing biodistribution of RVG-EVS, G58TF-RVG-EVs and G58T/siRNA-RVG-EVs. Images were taken after 4 h of systemic administration of EVs. Surface proteins of RVG-EVs were labeled with cy5.5-NHS fluorescent dye. Mice injected with saline was used as a negative control. Histogram at the right side shows quantification of the fluorescent signal from the brain of treated mice. Results shown as mean±s.d. **p=0.001 and ***p<0.0001 compared to control (saline) group (One way ANOVA; n=3). (e) In vivo silencing of HTT gene in Q140 HD mouse model after systemic administration of G58TF EVs. A total of four doses of G58TF-RVG-EVs were injected into animals. Doses were given weekly. After 72 h after of the last dose, animals were sacrificed and different sections of brain were analysed for HTT mRNA level, using probe-based qRT-PCR. Data were normalized with 18S rRNA. Animals receiving saline were used as control to determine HTT level in Neg. siRNA group (animals that received G58TF EVs+neg. siRNA) and HTT siRNA group (animals that received G58TF+HTT siRNA). Results shown as mean±s.d. ns: non-significant, **p=0.0012 when compared to control group. (One Way ANOVA; n=6) (f) Immunohistochemistry of cortex regions of the brain from the sacrificed animals. Images show mutant HTT protein level and p62-labeled inclusion bodies in the neurons of the cortex of HTT siRNA and Neg. siRNA treated animals. Histogram at the right shows quantification of p62 inclusion bodies in HTT siRNA and Neg. siRNA treated groups. Results are shown as mean±s.d, n=8 slides chosen randomly from each group. **p=0.003 when compared to neg. siRNA group (Two way ANOVA)

FIG. 4 GAPDH present on the outer surface of EVs binds to cleaved lactoferrin

(a) Protease digestion assay of EVs under native and denatured conditions. Top left represents schematic of protease digestion assay. CD81-GFP was used as positive control for protein resided in the lumen of EVs. After protease treatment, lactoferrin, GFP and GAPH proteins were analyzed by western blotting as shown on the right side of the figure. Lane 1 represents standard protein marker, Lane 2 represents non-treated EVs. Lane 3, 5, 7 and 9 represents EVs incubated with protease at the indicated time points under native conditions. EVs in lane 4, 6, 8 and 10 were denatured to dissolve the membrane and then treated with protease for the given amount of time. Assay was carried out at 37° C. Digestion of lactoferrin and GAPDH proteins under native condition confirms their presence on outer surface of EVs. Presence of intact GFP band after incubating EVs till 60 min with protease under native conditions confirms integrity of the membrane during the treatment. (b) Western blots of EVs from different cells after protease digestion assay. Digestion of GAPDH protein under native and denatured condition confirms presence of GAPDH on outer surface of EVs. CD81-GFP and CD81-TRBP were used as positive control for proteins present in the lumen of the EVs. (c) GAPDH enzyme kinetics of EVs from different cell sources. Equal number of EVs were used to monitor in real time the formation of NADH by using KDalert GAPDH assay kit (Invitrogen). Values are the fluorescence intensity of NADH measured under kinetic mode at λ abs. of 560 nm and λ emi. Of 590 nm. Data was normalized with the blank sample, containing substrate only. Results shown as mean±s.d, N=3 (d) Co-immunoprecipitation of lactoferrin and GAPDH protein expressed in HEK293T cells and EVs. Beads conjugated to cMyc antibody were used to capture GAPDH-cMyc protein in the cell lysate that were analyzed by western blotting, using lactoferrin antibody. LG; lactoferrin-GFP protein, LGS; Lactoferrin-GFP with signal peptide of LAMP-2B, WCL; crude cell lysate used as positive control. Presence of a-tubulin bands shows interaction of GAPDH with a-tubulin. Insulin triphosphate receptor 3 (IPR3), Argonuate 2 (Ago2) and mitochondrial import receptor subunit TOM20 were used as negative controls. Western blot Image on the right side shows co-immunoprecipitation of lactoferrin and GAPDH on surface of EVs. cMyc beads were used to capture GAPDH cMyc protein from EVs. Captured proteins were analyzed by western blotting using GFP, lactoferrin and GAPDH antibody. Western blotting image shows presence of lactoferrin-GFP and lactoferrin-LAMP-2B bands, confirming their interaction with GAPDH proteins. (e) Chromatogram of gel filtration chromatography showing absorbance of EVs at 280 nm after incubated with Lactoferrin N1.1 (lacN1.1) and Lactoferrin N (LacN) respectively. The protein was labelled with alexa fluor-633. Inset represents EVs bound to alexa fluor-633 labelled lactoferrin N protein.

FIG. 5 Binding of Exogenous GAPDH to EVs

(a) Co-immunoprecipitation assay showing interaction of different domains of lactoferrin N protein with GAPDH. Different domain of the lactoferrin N are shown as schematic at the top of the figure. Domains were fused to GFP and expressed in HEK293T cells. cMyc beads were used to capture GAPDH-cMye proteins. Lactoferrin N2 domain showed interaction with the GAPDH. Representative image (n>3). (b) Exogenous binding of GAPDH to EVs isolated from the different cells. (c) GAPDH kinetic assay of EVs before and after binding to exogenous GAPDH. Equal no. of EVs were added to 96 well-plates in triplicates. KDalert GAPDH assay kit was used to determine the GAPD kinetics. Results shown as mean±s.d, n=3. (d) UV-absorbance spectrum of EVs after passing through gel-filtration column. Crude EVs present in the concentrated tissue culture media were incubated with 1 mg of GAPDH protein for 2 h at 4° C. and passed through the gel filtration column. Increase in the OD280 is due to binding of GAPDH to EV surface and possibly aggregation of GAPDH bound EVs. First peak (around 10 ml elution) represent EVs and second peak (around 20 ml elution) represent tissue culture media protein Representative image (n>3)

FIG. 6 G58T quantification and Drosophila GAPDH binding to human EVs

(a) Western blot showing quantification of G58T protein on MSCs and 293T EVs. 6.75 E+11 EVs were incubated with 22 nmoles of G58T protein for 2 h at 4° C. Excess of unbound protein was separated by gel-filtration chromatography. A given amount of purified G58T protein was run on SDS PAGE along with EVs samples to determine the number of G58T proteins on EVs. (b) Linear regression analysis of above immunoblots quantified using image studio software of LI-COR. (c). Scatter plot showing number of G58T proteins bound to each MSCs and 293T EVs as determined by quantification of immunoblots. Data shown as mean±s.d. n=3 (samples run in duplicates). (d) western blots of MSCs and 293T EVs after incubating with Drosophila GAPH. 50 ul of 2 mg/mL BSA was added during the incubation. Representative blot (n=2). (e) Nanosight tracking analysis of EVs after incubation with Drosophila GAPDH protein. 20 nmoles of GAPDH was incubated with 1.0E+12 number of EVs for 2 h. A change in size of EVs after binding to GAPDH2 reflects clumping of EVs catalysed by Drosophila GAPDH. Representative graph (n=3).

FIG. 7 G58 mediated loading of siRNA into EVs

(a) SDS-PAGE of the proteins purified by Ni-NTA chromatography. Gel is stained with Coomassie brilliant blue dye. (b) Gel shift assay of G58TEVs (upper) and G58TF EVs (lower), reflecting binding of siRNA to EVs. 20 pmoles of siRNA was incubated with the given number of EVs. Bands in the wells represent bound siRNA. siRNA alone was used as negative control. Based on gel shift assay, around 550 siRNA are binding per G58T EVs and 714 siRNA are binding to each G58TF EVs. Higher binding of siRNA to G58TF is due to arginine rich FHV peptide. (c) RNase A protection assay of G58TF EVs bound to 50 pmoles of siRNA. Bands in the agarose gel represent siRNA isolated from G58TF EVs after treatment with RNase A (0.2 mg/ml). G58TF EVs bound to siRNA were incubated with RNase A for 6 h at 37° C. (d) Representative confocal microscopy images of N2a cells after 4 h of treatment with G58TF EVs carrying Cy-3 labelled siRNA (green). Surface proteins of EVs were labelled with alexa fluor-633 (red). Nuclei of cells is stained with Hoechst (blue). Inset in the merged figure represents magnified image of N2a cells showing colocalization of siRNA and EVs (yellow spots). Images were captured using 60× objective lens of Olympus confocal microscope FV1000. (e) HTT gene silencing at mRNA level by G58TF EVs in N2a cells after 48 h of treatment. Results are shown as mean±s.d, N=3 (***p<0.0001 was considered statistically significant by using non-parametric one way ANOVA to compare means difference among treated and non-treated cells) (f) Western blot showing GAPDH silencing in HeLa cells by G58TF EVs in presence and absence of chloroquine (30 μM). 40 pmoles of siRNA bound to EVs were added to cells and incubated for 72 h. (g) In vivo animal imaging of C57BL/6 animals after 4 h of administration of G58TF EVs labelled with cy-5.5 dye. Images represent fluorescence of cy-5.5 dye in various organs of the animals. Modification of EV surface with G58TF and siRNA did not alter biodistribution of RVG EVs. Three animals were used in each group. Graph shows quantification of the signals in different organs of the animals. Results are shown as mean±s.d.

FIG. 8 Biodistribution of EVs in C57BL/6 mice

In vivo fluorescence images of C57BL/6 mice brain showing biodistribution of RVG-EVS, G58TF-RVG-EVs and G58T/siRNA-RVG-EVs. Images were taken after 4 h of systemic administration of EVs. Surface proteins of RVG-EVs were labeled with cy5.5-NHS fluorescent dye.

FIG. 9 Size of EVs after incubation with GAPDH protein

Electron microscopy images of EVs before and after incubation with GAPDH. Incubation of EVs with GAPDH caused fusion of EVs. In other words, the formation of long chains of aggregation of EVs incubated with GAPDH for 2 h at 4° C. 1×10¹² EVs were incubated with GAPDH at 1:10,000 particle ratio.

FIG. 10 Ribbon diagram of human GAPDH

Top—Ribbon diagram of human GAPDH showing tetramerization of GAPDH protein. Bottom—Design of G58-dsRBP fusion protein to determine binding of G58-dsRBP to EV surface. An EV may bind to each tetramer, resulting in aggregation of EVs. GD1 relates to the GAPDH monomer in the top left quadrant.

FIG. 11 Design of G58-dsRBP fusion proteins for EV-mediated gene silencing

Schematic representation of different G58 fusion protein for delivery of siRNA into cells and tissues. Different peptide tag have been attached to enhance release of siRNA from late endosomes (a). Gel binding assay of G58 modified EVs to determine binding of siRNA to EVs (b).

FIG. 12 Conjugation of G58 peptide to magnetic beads for purification of extracellular vesicles

Surface protein of EVs were conjugated to Cy5.5 fluorescent dye to determine the binding of the EVs. Given number of the EVs were incubated with the magnetic beads for 2 h and isolated by pulling the beads via magnetic bar. Histogram shows the number of EVs before and after incubation with the G58T conjugated magnetic beads. EVs were counted using nanosight tracking analysis system. Inset represent Cy5.5 labelled EVs before and after incubation with G58T conjugated magnetic beads. Reduction in the intensity of cyan colour indicates binding of the EVs to magnetic beads. B.B: Before binding; A.F: After binding.

FIG. 13 GAPDH binds to EV surface via G58 domain a Western blot showing binding of G58 peptide to HEK293T (designated as 293T) and MSC EVs. The second domain of TARBP protein was attached to G58 peptide for detection by anti-TARBP2 antibody. b NTA profile showing the size distribution of HEK293T EVs after binding to the G58T protein. Inset is the scatter plot representing size (mean) of EVs. Data are shown as mean±s.d, n=9 (ns=non-significant, two tailed t-test). c Gel-shift assay of EVs after incubation with either G58T (G58+dsRBD) protein or dsRBD of TARBP2 protein. siRNA alone was used as negative control to determine interaction of EVs with siRNA. A gradual decrease in the intensity of siRNA reflects entrapment of siRNA near the wells due to interaction with G58T EVs. Lack of siRNA binding to dsRBD treated EVs confirms G58 peptide mediated binding of protein to EV surface. d-f High resolution single EV analysis by Imaging Flow Cytometry (IFC) to determine localization of GAPDH and G58 peptide on EVs. d Represents method validation by using either non-labelled HEK293F derived EVs or neon GFP labelled HEK293:CD63-neon GFP derived EVs as biological reference material. e Detection of GAPDH on HEK293F, HEK293F/CD63-GFP and MSCs EVs, using alexa fluor 647 labelled anti-GAPDH antibody. f G58 peptide binding on EVs expressing GAPDH on their surface. EVs were incubated with alexa fluor 488 (af488) labelled G58 peptide and af647 anti-GAPDH antibody. g Distribution of secreted GAPDH-GFP protein in the cell-culture media. Media from HEK293T cells expressing GAPDH-GFP protein were processed to isolate EVs from proteins by gel-filtration chromatography. Both EVs and protein fractions contained GAPDH-GFP protein, indicating vesicular and non-vesicular modes of GAPDH secretion. Data shown as mean±s.d, n=3.

FIG. 14 Manipulating GAPDH levels in Drosophila secondary cells affects the biogenesis of CD63-GFP-labelled exosomes

a Schematic shows isoforms of Drosophila GAPDH1 and GAPDH2 and the targeted regions of each RNAi line used. Except for the gapdh2-RNAi #2, these RNAi lines do not have predicted off-targets. The two major protein domains, the highly conserved catalytic domain (green line) and the NAD binding domain (yellow, shorter line), are also shown. b Basal wide-field fluorescence and differential interference contrast (‘Merge’) views of living secondary cells (SCs) expressing GFP-tagged form of CD63 (CD63-GFP; green) with no other transgene (control); or also expressing the open reading frame of the human GAPDH protein (hGAPDH), two independent RNAi constructs targeting Drosophila GAPDH1 (gapdh1—RNAi #1 and #2), or two independent RNAi constructs targeting Drosophila GAPDH2 (gapdh2—RNAi #1 and #2) from eclosion onwards. SC outline approximated by dashed white circles, and acidic compartments are marked by LysoTracker Red (magenta). CD63-GFP-positive intraluminal vesicles (ILVs; green in ‘Merge’; grey in ‘Zoom’) are apparent inside compartments, surrounding dense-core-granules (DCGs; asterisk in ‘Zoom’) and connecting DCGs to the limiting membrane of the compartment (yellow arrowheads, except in GAPDH2 knockdown, where ILVs only surround peripheral small DCGs). DCG compartment outline is approximated by white circles. Panel also shows confocal transverse images of fixed accessory gland (AG) lumens from the same genotypes, containing CD63-GFP fluorescent puncta. c Bar chart shows average number of large (>1 μm diameter) CD63-GFP-positive compartments per cell. d Bar chart shows average number of large (>1 μm diameter) Btl-GFP-positive compartments per cell. e Bar chart shows the percentage of CD63-GFP-positive compartments per cell containing ILVs. f Bar chart (n=10) shows number of CD63-GFP fluorescent puncta in the lumen of AGs for the different genotypes. Clustering of exosomes in the presence of hGAPDH prevented accurate quantification. g Basal confocal images of fixed SCs (n=4) isolated from males expressing Btl-GFP with no other transgene (control), or also expressing hGAPDH from eclosion onwards. hGAPDH (magenta) and DAPI (blue) staining are shown. SC outline approximated by dashed white circles. GAPDH appears to associate with membranous structures inside late endosomal and lysosomal compartments when hGAPDH is overexpressed (yellow arrowheads in ‘Zoom’). All data are from six-day-old male flies shifted to 29° C. at eclosion to induce expression of transgenes. Genotypes are: w; P[w+, UAS-CD63-GFP] P[w+, tub-GAL80ts]/+; dsx-GAL4/+ with no other transgene (control), UAS-hGAPDH, UAS-gapdh1-RNAi #1 and #2 (BL #62216), UAS-gapdh2-RNAi #1 and #2. Scale bars in (b) (5 μm), in ‘Zoom’ (1 μm), and in ‘AG lumen’ (20 μm). ***P<0.001, **P<0.01 and *P<0.05 relative to control, n=23-27 cells.

FIG. 15 GAPDH2 knockdown affects exosome and DCG biogenesis in SCs, but not Rab11-compartment identity

a Basal wide-field fluorescence and differential interference contrast (‘Merge’) views of living secondary cells (SCs) expressing the YFP-Rab11 gene trap (YFP-Rab11; yellow) with no other transgene (control), or also expressing either of two independent RNAi constructs targeting Drosophila GAPDH2 (gapdh2—RNAi #1 and #2) from eclosion onwards. SC outline approximated by dashed white circles, and acidic compartments are marked by LysoTracker Red (magenta). YFP-Rab11-positive intraluminal vesicles (ILVs; yellow in ‘Merge’; grey in ‘Zoom’) are apparent inside compartments, but only near the compartment's limiting membrane in GAPDH2 knockdown cells (yellow arrowheads). DCG compartment outline is approximated by white circles. b Bar chart showing the percentage of ILV-containing large (>1 μm diameter) compartments per cell marked with CD63-GFP. Btl-GFP or YFP-Rab11. c Bar chart showing the percentage of DCG compartments per cell containing a fragmented DCG. d Bar chart showing the percentage of DCG compartments per cell containing an abnormally shaped DCG. All data are from six-day-old male flies shifted to 29° C. at eclosion to induce expression of transgenes. Genotypes are: w; P[w+, UAS-CD63-GFP] P[w+, tub-GAL80ts]/+; dsx-GAL4/+ with no other transgene (control #1), w; P[w+, tub-GAL80ts]/+; dsx-GAL4, P[w+, UAS-btl-GFP]/+ with no other transgenes (control #2), w; P[w+, tub-GAL80ts]/+; dsx-GAL4, TI{TI}Rab11EYFP/+ with no other transgene (control #3), or the same genotypes with UAS-gapdh2-RNAi #1 and #2. Scale bars in (a) (5 μm) and in ‘Zoom’ (1 μm). Data shown as mean±s.d., ***P<0.001, **P<0.01 and *P<0.05 relative to control, n=26-33 cells.

### DETAILED DESCRIPTION OF THE INVENTION

The present disclosure is directed to EVs and to an EV-binding moiety-mediated loading of EVs with cargo. The EV-binding moiety may be selected from a lipid and/or an EV protein. The EV-binding moiety may be a PS-binding protein that mediates loading of EVs with cargo. The EV-binding moiety may be any protein, peptide or aptamer that bind to the same site as GAPDH's G58 domain. The present disclosure is also directed to EVs and to GAPDH-mediated loading of EVs with cargo. Loaded EVs may be used for targeted and non-targeted delivery of the EV and/or the EV cargo. The PS-binding protein moiety may be selected from one or more of a number of PS-binding protein and/or peptide, including a PS-binding variant or fragment thereof. The PS-binding protein moiety may be GAPDH and/or a PS-binding variant or fragment thereof. The protein bound to EV may be GAPDH variant or fragment thereof. Alternatively, the PS-binding protein moiety may be selected from a PS-binding protein and/or peptide that is not GAPDH, is not an annexin such as annexin A1, A2, A3, A4, A5, A6, A7, A8, A8L1, A8L2, A9, A10, A11 or A13, is not factor VIII, is not lactadherin, and/or is not a variant or fragment thereof. The PS-binding protein moiety may bind to PS substantially independently of the concentration of Ca2⁺.

The inventors demonstrate a novel role for GAPDH, a glycolytic enzyme, in the secretion of EVs and exploit these findings to develop a GAPDH-based methodology to load cargo onto EVs, for example for targeted delivery to cells, tissues and organs, such as the brain. The inventors observed high levels of GAPDH binding to the outer surface of EVs via a motif, designated as G58, and discover that the enzyme's tetrameric nature promoted extensive EV aggregation. Studies in a Drosophila EV biogenesis model define that GAPDH is required for normal intraluminal vesicle formation in endosomal compartments and promotes clustering of vesicles both inside and outside the cell. By fusing the GAPDH derived G58 peptide to dsRNA-binding motifs, the inventors showed that cargo, such as RNA-based drugs like siRNA can be loaded onto EVs. Such loaded EV efficiently delivered their cargo to the target cells in vitro and in vivo, such as into the brain of a Huntington's disease mouse model, resulting in silencing of the huntingtin gene in multiple anatomical brain regions. Thus, the inventors demonstrate a novel role of GAPDH in EV biogenesis, and that the presence of free GAPDH binding sites on EVs can be effectively exploited to substantially enhance the therapeutic potential of EVs in drug delivery. The inventors have developed a simple and robust method for loading cargo such as RNA-based drugs onto EVs, for example for targeted delivery.

Extracellular Vesicles (EVs)

The terms “extracellular vesicle” or “EV” or “exosome” shall be understood to relate to any type of vesicle that is, for instance, obtainable from a cell, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), ARRDC1 Mediated Microvesicle (ARMM), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. The EV may be a natural vesicle that is secreted by a cell and/or produced by an individual. Furthermore, the said terms shall be understood to also relate to in some embodiments extracellular vesicle mimics, cellular membrane vesicles obtained through membrane extrusion or other techniques, etc. Essentially, the present invention may relate to any type of lipid-based structure (with vesicular morphology or with any other type of suitable morphology) that can act as a delivery or transport vehicle for the ubiquitin ligase, and optionally an antibody. It will be clear to the skilled artisan that when describing medical and scientific uses and applications of the EVs, the present invention normally relates to a plurality of EVs, i.e. a population of EVs which may comprise thousands, millions, billions or even trillions or even more EVs. In the same vein, the term “population” shall be understood to encompass a plurality of entities which together constitute such a population. In other words, individual EVs when present in a plurality constitute an EV population. Thus, naturally, the present invention pertains both to individual EVs and populations of EVs, as will be clear to the skilled person. Similar reasoning naturally applies to the genetically modified cells of the present invention, i.e. that the invention relates to both individual cells and populations of such cells.

Extracellular vesicles (EVs) are lipid bilayer-delimited particles that are naturally released from a cell and, unlike a cell, cannot replicate. EVs range in diameter from near the size of the smallest physically possible unilamellar liposome (around 20-30 nm) to as large as 10 μm. Thus, the EVs can have a diameter of 30 nm-150 nm, 30 nm-250 nm, 30 nm-500 nm, 30 nm-1000 nm, 150 nm-250 nm, 150 nm-500 nm, 150 nm-1000 nm, 250 nm-500 nm, 250 nm-1000 nm or 500 nm-1000 nm. EVs can carry a variety of cargo, such as proteins, nucleic acids, lipids, metabolites, small molecule drugs, biological drugs such as antibodies, and/or organelles from the parent cell. Most cells that have been studied to date release EVs, including eukaryotic cells such as animal and plant cells, bacterial cells and fungal cells. In addition, EVs have also been isolated from physiological fluids, such as plasma, urine, amniotic fluid and malignant effusions. A wide variety of EV subtypes have been proposed, defined variously by size, biogenesis pathway, cargo, source, and function. EVs for use in accordance with the present invention can be derived from any suitable cell or physiological fluid.

During the last decade, a new paradigm of cell-to-cell communication has emerged involving EVs, with implications for both normal and pathological physiology. Given their unique biological and pharmacological characteristics, EVs have gained tremendous interest in understanding their roles in physiology, pathophysiology and drug delivery. Immunological inertness, ability to cross biological barriers, and to carry bioactive molecules are some of the attractive features of EVs that can be exploited in drug delivery applications. However, lack of efficient drug loading methods, and an incomplete understanding of EV biogenesis and uptake mechanisms remain critical challenges that need to be addressed. Current methods of loading therapeutic molecules into EVs such as electroporation, genetic engineering of host cells and chemical conjugation, are limited by low efficiency, toxicity and lack of scalability. Moreover, they produce a heterogeneous population of EVs that imposes complexity in understanding phenotypic effect of EVs in the targeted cells. To harness therapeutic potential of EVs, it is important to understand intracellular pathways of EV biogenesis, which will provide opportunities to exploit the natural characteristics of EVs for therapeutic applications. There is a growing interest in clinical applications of EVs as biomarkers and therapies alike.

The EV may be an exosome. Exosomes are produced in the endosomal compartment of most eukaryotic cells. The multivesicular body (MVB) is an endosome defined by intraluminal vesicles (ILVs) that bud inward into the endosomal lumen. If the MVB fuses with the cell surface (the plasma membrane), these ILVs are released as exosomes. In multicellular organisms, exosomes and other EVs are present in tissues and can also be found in biological fluids including blood, urine, and cerebrospinal fluid. They are also released in vitro by cultured cells into their growth medium. Since the size of exosomes is limited by that of the parent MVB, exosomes are generally thought to be smaller than most other EVs, from about 20 to several hundred nm in diameter: around the same size as many lipoproteins but much smaller than cells.

EVs may form aggregates. Such aggregates arise through covalent and/or non-covalent interactions between molecules on the surface of the EV that result in two or more discrete EVs associated with each other such that the associated EVs substantially move together as one unit when in bulk solution. In a preferred embodiment, aggregation of EVs is minimized or eliminated, such that EVs exist as substantially discrete entities.

In addition, the present inventors showed that GAPDH binding sites are present on the inner and outer surfaces of the EV membrane. Such GAPDH binding sites were observed on EVs isolated from various different sources, including different cellular sources. The inventors also showed that these GAPDH binding sites can be used to successfully load cargo onto EVs.

Cell Sources

Generally, EVs may be derived from essentially any cell source, be it a primary cell source or an immortalized cell line. The EV source cells may be any embryonic, foetal, and adult somatic stem cell types, including induced pluripotent stem cells (iPSCs) and other stem cells derived by any method, as well as any adult cell source. The source cells per the present invention may be select from a wide range of cells and cell lines, for instance mesenchymal stem or stromal cells (obtainable from e.g. bone marrow, adipose tissue, Wharton's jelly, perinatal tissue, chorion, placenta, tooth buds, umbilical cord blood, skin tissue, etc.), fibroblasts, amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, erythrocytes, erythroid progenitors, chondrocytes, MSCs of different origin, amnion cells, amnion epithelial (AE) cells, any cells obtained through amniocentesis or from the placenta, airway or alveolar epithelial cells, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein. When treating neurological diseases, one may contemplate to utilise as source cells e.g. primary neurons, astrocytes, oligodendrocytes, microglia, and neural progenitor cells. The source cell may be either allogeneic, autologous, or even xenogeneic in nature to the patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a patient suffering from a certain indication. For instance, in the context of treating systemic, peripheral and/or neurological inflammation, allogeneic MSCs or AEs may be preferable as EVs obtainable from such cells may enable immuno-modulation via e.g. macrophage and/or neutrophil phenotypic switching (from pro-inflammatory M1 or N1 phenotypes to anti-inflammatory M2 or N2 phenotypes, respectively). The most advantageous source cells per the present invention are MSCs, amnion-derived cells, amnion epithelial (AE) cells, any perinatal cells, and/or placenta-derived cells, all of which are of mammal, most preferably of human, origin. The cell lines from which EVs are derived may be adherent or suspension cells and may be generated as stable cell lines or single clones.

Phosphatidylserine- (PS-) Binding Protein

A Phosphatidylserine- (PS-) binding protein is any protein and/or peptide that binds the lipid phosphatidylserine (PS). Alternatively, or in addition, the PS-binding protein and/or peptide may bind to a lipid that is not PS and/or to an EV protein. Where binding of a PS-binding protein and/or peptide to a lipid that is not PS and/or to an EV protein is in addition to binding to PS, the additional binding to the lipid that is not PS and/or to the EV protein may facilitate and/or enhance binding to the PS-binding protein and/or peptide to PS and/or to the EV.

The PS-binding protein and/or peptide may be expressed recombinantly in the host cell from which the EV is isolated from. Alternatively, or in addition, the PS-binding protein may expressed recombinantly in a separate cell and added to an isolated EV.

PS may be present in the inner and/or outer lipid bilayer of an EV. A PS-binding protein may associate with an EV through an interaction with PS and thus may be present on the surface and/or interior or the EV. The interaction between that PS-binding protein and PS may be through a non-covalent interaction. Examples of non-covalent interactions include electrostatic interactions such as ionic interactions, hydrogen bonding and halogen bonding. Examples of non-covalent interactions also include Van der Waals forces and hydrophobic effects.

The PS-binding protein may be selected from one or more of annexin, copine, DGK, DOC 1, DOC2, dynamin, erythrocyte protein 4.1, factor V, factor VII, factor VIII, factor IX, factor X, FGF, GAPDH, gas-6, lactadherin, MARCKS, neutral sphingomyelinase, Na/K ATPase, NO synthase, PKC, PLC, protein C, protein S, prothrombin, phosphatidylserine receptor, rabphilin, Raf-1, scavenger receptor, SK1, synaptotagmin and vinculin, and/or a phosphatidylserine-binding variant or fragment of anyone thereof. In some embodiments, the PS-binding protein is not GAPDH, is not an annexin such as annexin A1, A2, A3, A4, A5, A6, A7, A8, A8L, A8L2, A9, A10, A11 or A13, is not factor VIII, is not lactadherin, and/or is not a variant or fragment thereof. In some embodiments, the PS-binding protein is GAPDH or a variant or fragment thereof.

The GAPDH protein or variant or fragment thereof may comprise:
(a) a polypeptide sequence having at least 80%, at least 90%, at least 95% or at least 100% sequence identity to SEQ ID NO: 1, optionally comprising 1-10 additional amino acids at the 5′ and/or 3′ end;
(b) a polypeptide having at least 80%, at least 90%, at least 95% or at least 100% sequence identity to SEQ ID NO: 2; and/or
(c) at least 10, at least 20 or at least 30 contiguous amino acid residues from the polypeptide sequence of SEQ ID NOs: 1 or 2.

The number of PS-binding protein molecules associated with each EV may be 1-10, 1-100, 1-500, 1-1000, 1-3000, 1-5000, 1-10,000, 100-500, 100-1000, 100-3000, 100-5000, 100-10,000, 500-1000, 500-3000, 500-5000, 500-10,000, 1000-3000, 1000-5000, 1000-10,000, 3000-5000, 3000-10,000 or 5000-10,000. The number of PS-binding protein molecules associated with each EV may be at least about 10 molecules, at least about 100 molecules, at least about 500 molecules, at least about 1000 molecules, at least about 3000 molecules, at least about 5000 molecules or at least about 10,000 molecules. A particular advantage of certain embodiment of the invention is the presence of at least about 500 PS-binding proteins associated with each EV. The number of PS-binding proteins associated with each EV according to the invention is preferably increased compared to the number of PS-binding proteins associated with each EV in a wild type setting. In this context, a wild type setting may refer to an unmodified naturally occurring EV and/or an unmodified naturally occurring PS-binding protein. The number of PS-binding proteins associated with each EV according to the present invention may be increased, when compared to a wild type setting, by at least 1.25 fold, at least 1.5 fold, at least 2 fold, at least 5 fold, at least 10 fold, at least 25 fold, at least 50 fold, at least 100 fold, at least 250 fold, at least 500 fold, at least 1000 fold, at least 2500 fold, at least 5000 fold, or at least 10,000 fold.

Of the molecules of PS-binding protein associated with the EV, the EV may comprise a single type of PS-binding protein, two different types of PS-binding protein, three different types of PS-binding protein, four different types of PS-binding protein, five different types of PS-binding protein, six different types of PS-binding protein, seven different types of PS-binding protein, eight different types of PS-binding protein, nine different types of PS-binding protein, ten different types of PS-binding protein or more than ten different types of PS-binding protein. In other words, the EV may comprise of a homogenous combination of PS-binding proteins or a heterogeneous combination of PS-binding proteins.

As set out above, the PS-binding protein and/or peptide may bind to a lipid that is not PS and/or an EV protein. Non-limiting examples of lipids that the PS-binding protein and/or peptide may bind to include phospholipids, glycolipids, fatty acids, phosphoglycerides, sphingolipids and sterols such as cholesterol. Examples of phospholipids include ceramide phosphorylcholine, ceramide phosphorylethanolamine, ceramide phosphoryllipid, phosphatidic acid, phosphatidylethanolamine, phosphatidylcholine, phosphatidylserine, and phosphoinositides such as phosphatidylinositol, phosphatidylinositol phosphate, and phosphatidylinositol bisphosphate and phosphatidylinositol trisphosphate, and combinations, derivatives, variants, or regions thereof. The lipid may be present in the membrane of the EV. Non-limiting examples of EV proteins that the PS-binding protein and/or peptide may bind to include CD9, CD53, CD63, CD81, CD54, CD50, FLOT1, FLOT2, CD49d, CD71, CD133, CD138, CD235a, ALIX, Syntenin-1, Syntenin-2, LAMP-2B, TSPAN8, syndecan-1, syndecan-2, syndecan-3, syndecan-4, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, LICAM, LAMB1, LAMC1, ARRDC1, PDGFRN, ATP2B2, ATP2B3, ATP2B4, BSG, IGSF2, IGSF3, IGSF8, ITGB1, ATPlA2, ATPlA3, ATPIA4, ITGA4, SLC3A2, ATP1A1, ATP1B3, ATP2B1, LFA-1, LGALS3BP, Mac-1 alpha, Mac-1 beta, MFGE8, SLIT2, STX3, TCRA, TCRB, TCRD, TCRG, VTI1A, VTI1B, and any other EV proteins, and any combinations, derivatives, domains, variants, mutants, or regions thereof. The EV protein may be present in the membrane of the EV. The EV protein may be an exosomal protein. The EV protein may be any protein or peptide that is naturally associated with at least one type of EV, such as any protein or peptide that is naturally associated with at least one type of exosome. If an EV protein is not naturally present on all EVs, it may still be introduced to an EV that does not naturally contain the protein and said EV containing the introduced protein used according to the present invention.

Proteins and Variants and Fragments Thereof

References to proteins, peptides and polypeptides are used interchangeably within the present disclosure. Peptides that can bind to the inner and/or outer surface of an EV are disclosed. Such peptides may bind to one or more molecule present in the membrane of an EV. The one or more molecule present in the membrane of an EV may be a protein, and/or a lipid such as cholesterol and/or PS. As set out in this disclosure, peptides that can bind to the inner and/or outer surface of an EV have utility in loading cargo onto an EV and purifying an EV. Peptides that can bind to the inner and/or outer surface of an EV also have utility as in vitro and/or ex vivo research tools, diagnostic tools, imaging tools, biological reference material, an experimental control and/or an experimental reference standard.

An example of a peptide that can bind to the inner and/or outer surface of an EV is GAPDH or a variant or fragment thereof that retains the ability to bind to the surface of an EV. The variant or fragment of GAPDH may be a peptide termed G58 and/or G70. The present inventors showed that G58 and G70 mediates binding of GAPDH to the surface of EVs. The GAPDH protein or variant or fragment thereof may comprise:
(a) a polypeptide sequence having at least 80%, at least 90%, at least 95% or at least 100% sequence identity to SEQ ID NO: 1, optionally comprising 1-10 additional amino acids at the 5′ and/or 3′ end;
(b) a polypeptide having at least 80%, at least 90%, at least 95% or at least 100% sequence identity to SEQ ID NO: 2; and/or
(c) at least 10, at least 20 or at least 30 contiguous amino acid residues from the polypeptide sequence of SEQ ID NOs: 1 or 2.

An example of a peptide that can bind to the inner and/or outer surface of an EV is a PS-binding protein. The PS-binding protein may be selected from one or more of annexin, copine, DGK, DOC 1, DOC2, dynamin, erythrocyte protein 4.1, factor V, factor VII, factor VIII, factor IX, factor X, FGF, GAPDH, gas-6, lactadherin, MARCKS, neutral sphingomyelinase, Na/K ATPase, NO synthase, PKC, PLC, protein C, protein S, prothrombin, phosphatidylserine receptor, rabphilin, Raf-1, scavenger receptor, SK1, synaptotagmin and vinculin, and/or a phosphatidylserine-binding variant or fragment of anyone thereof.

Purification and/or Identification of an EV

The interaction between peptides of the present disclosure and EVs, and in particular the surface of an EV, can be used to purify and/or to identify an EV. For example, a peptide that binds to an EV may be used to capture an EV in bulk solution through its interaction with an EV, in accordance with methods known in the prior art. The peptide that binds to an EV may be immobilised on a solid support, for example on a bead such as a magnetic bead, or on the surface of a 96-well plate. The captured EV may then be eluted from the peptide and/or the solid support, for example by altering the salt concentration, altering the pH, and/or washing with a fluid such as glycerol. Peptides used to capture an EV may comprise a single type of peptide, two different types of peptides, three different types of peptides, four different types of peptides, five different types of peptides, six different types of peptides, seven different types of peptides, eight different types of peptides, nine different types of peptides, ten different types of peptides, or more than ten different types of peptides. In other words, the peptides used to capture an EV may be homogenous, or heterogonous.

By capturing an EV in this way, it is possible to purify an EV from bulk solution. It is also possible to use the peptide that binds to an EV as a diagnostic tool such as in an ELISA assay, an imaging tool for example by conjugating the peptide to a fluorophore, a biological reference material, an experimental control and/or an experimental standard.

The peptide that binds to an EV may itself be bound to a different EV, such than an EV composition is used as the purification agent, diagnostic agent, research tool, imaging tool, biological reference material, experimental control and/or experimental standard.

Variants and Fragments

The following section relates to general features of all proteins and/or peptides (i.e. polypeptides), and in particular to variations, alterations, modifications, fragments or derivatisations of amino acid sequence. It will be understood that such variations, alterations, modifications fragments or derivatisations of proteins and/or peptides as are described herein are subject to the requirement that the proteins and/or peptides retain any further required activity or characteristic as may be specified other sections of this disclosure, such as PS-binding activity and/or EV-binding activity.

Variants of proteins and/or peptides may be defined by particular levels of amino acid identity which are described in more detail in subsequent sections of this disclosure. Amino acid identity may be calculated using any suitable algorithm. For example the PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.nebi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate homology (for example used on its default settings) (Devereux et al (1984) Nucleic Acids Research 12, 387-395).

It will be understood that variants of proteins and/or peptides also includes substitution variants. Substitution variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

The amino acid sequence of proteins and/or peptides for use in the invention may be modified to include non-naturally occurring chemistries or to increase the stability and targeting specificity of the compound. When the proteins and/or peptides are produced by synthetic means, such amino acids may be introduced during production. The proteins and/or peptides may also be modified following either synthetic or recombinant production.

A number of side chain modifications are known in the art and may be made to the side chains of the proteins and/or peptides, subject to the proteins and/or peptides retaining any further required activity or characteristic as may be specified herein.

Variant proteins and/or peptides as described in this section are those for which the amino acid sequence varies from that in SEQ ID NO: 1 and/or SEQ ID NO: 2, but which retain the ability to bind PS.

The variant sequences typically differ by at least 1, 2, 3, 5, 10, 20, 30, 40 or more mutations (which may be substitutions, deletions or insertions of amino acids). For example, from 1 to 10, 2 to 5, or 3 to 20 amino acid substitutions, deletions (giving rise to a fragment) or insertions may be made, provided the modified proteins and/or peptide retains its activity, such as PS-binding activity. The amino acid substitutions, deletions or insertions may be contiguous or non-contiguous.

Typically, proteins and/or peptides which are variants of a PS-binding proteins and/or peptide have more than about 50%, 55% or 65% identity, preferably at least 70%, at least 80%, at least 90% and particularly preferably at least 95%, at least 97% or at least 99% identity, with the amino acid sequence of SEQ ID NO: 1. The identity of variants of SEQ ID NO: 1 and/or of SEQ ID NO: 2 may be measured over a region of at least 10, 20, 30, 40 or more contiguous amino acids of the sequence shown in SEQ ID NO: 1 or SEQ ID NO; 2, or more preferably over the full length of SEQ ID NO: 1 or SEQ ID NO: 2, excluding any signal sequence.

EV Cargo

The EV may be loaded with cargo. The terms “load”, “loaded”, “loading”, “onto an EV” and “into an EV” may be used in their broadest sense to describe a cargo associated with an EV such that the EV and its cargo move substantially together as one unit when in bulk solution. Thus, the cargo may be encapsulated in the interior (i.e. within the lumen) of the EV. Alternatively, or in addition, the cargo may be associated with the inner and/or outer lipid bilayer of the EV via a covalent and/or non-covalent interaction. However, in preferable embodiments, the cargo is associated directly and/or indirectly with the EV via a PS-binding protein that is itself associated with the EV.

The PS-binding protein and/or EV-binding protein may be linked to a second protein and/or peptide, and/or a small molecule drug. The second protein, peptide, and/or the small molecule drug may be fused to the PS-binding protein and/or EV-binding protein, for example through a covalent attachment. The second protein and/or peptide may be expressed recombinantly as a fusion protein with the PS-binding protein and/or the EV-binding protein in the host cell from which the EV is isolated from, or in a separate cell. The second protein, peptide, and/or the small molecule drug may be added to an isolated EV.

The second protein, peptide and/or small molecule drug may be selected from one or more of an enzyme, an antibody and/or antigen-binding variant or fragment thereof, a single chain variable fragment (scFv) and a cargo-binding protein and/or peptide. The cargo-binding protein and/or peptide may be selected from one or more of an antibody and/or antigen binding variant or fragment thereof, a single chain variable fragment (scFv), a nucleic acid-binding protein and/or peptide, and a nucleic acid analogue binding protein and/or peptide. The cargo-binding protein is any protein and/or peptide is any protein and/or peptide that can bind to a cargo of interest. The cargo-binding protein and/or peptide may be a RNA- and/or DNA-binding protein, for example a protein and/or peptide selected from one or more of TRBP2 and PKdsRBD2 and/or a RNA- and/or DNA-binding variant or fragment of anyone thereof.

The EV may be loaded with cargo, wherein the cargo binds to the PS-binding protein and/or peptide, and/or to the second protein and/or peptide. Binding may be through a covalent bond. Alternatively, binding may be through a non-covalent interaction. Examples of non-covalent interactions include electrostatic interactions such as ionic interactions, hydrogen bonding and halogen bonding. Examples of non-covalent interactions also include Van der Waals forces and hydrophobic effects. The cargo may be selected from one or more of a small molecule drug, a protein, a peptide, an antibody and/or antigen binding variant or fragment thereof, a single chain variable fragment (scFv), a nucleic acid, a nucleic acid analogue, gRNA, miRNA, shRNA, siRNA, piRNA, PMO and DNA. However, it will be apparent to the skilled person that an EV may be loaded with other types of cargo. Thus, the cargo to be loaded according to the present invention may be essentially any type of drug cargo, such as for instance mRNA, antisense or splice-switching oligonucleotides, siRNA, pDNA, supercoiled or unsupercoiled plasmids, mini-circles, peptides, proteins, antibodies, antibody-drug conjugates, gene editing technology such as CRISPR-Cas9, TALENs, meganucleases, or vesicle-based cargos such as viruses (e.g. AAVs, lentiviruses, etc.).

The present invention represents the first demonstration of the interaction between GAPDH, and variants and fragments thereof, and the membrane of EVs, can be used successfully to load cargo onto an EV, and in particular to load cargo onto the outer surface of an EV.

Genetic Material

It will be apparent that one specific type of cargo that can be loaded into EVs is genetic material such as nucleic acids. Nucleic acids are routinely used in gene therapy for the replacement of non-functional genes and for neutralization of disease-causing mutations via RNA interference (RNAi) effector molecules such as miRNAs, shRNAs and siRNAs. As naked DNA and RNA are difficult to deliver in vivo due to rapid clearance, nucleases, lack of organ-specific distribution and low efficacy of cellular uptake, specialized gene delivery vehicles, such as viral vectors and cationic liposomes, are usually used for delivery. Loading EVs with genetic material cargo according to the present invention has a number of advantages, such as overcoming mutagenic integration associated with viruses such as lentiviruses; and inflammatory toxicity and rapid clearance associated with liposomes. It may also be possible to reduce or eliminate recognition by the innate immune system and thus reduce or eliminate acute inflammatory responses associated with the naked delivery of genetic material.

The present inventors have successfully loaded EVs with exogenous genetic material, such as siRNA. Thus, the invention provides a composition comprising an EV, wherein the EV is loaded with genetic material cargo. The inventors have also shown that such loaded EVs have utility as gene delivery vehicles. The genetic material loaded into the EV may be genetic material that is typically associated with the EV and/or the host cell from which the EV is isolated, i.e. endogenous genetic material. Alternatively, the genetic material loaded into the EV may be genetic material that is typically not associated with the EV and/or the host cell from which the EV is isolated, i.e. exogenous genetic material. Thus, in one embodiment, an EV preparation that has already been isolated is loaded with genetic material

The genetic material may be modified. The genetic material may be single or double stranded. Single-stranded nucleic acids include those with phosphodiester, 2′O-methyl, 2′ methoxy-ethyl, phosphoramidate, methylphosphonate, and/or phosphorothioate backbone chemistry. Typically double-stranded nucleic acids are introduced including for example plasmid DNA and small interfering RNAs, such as siRNAs.

The genetic material to be loaded into the EVs is chosen on the basis of the desired effect of that genetic material on the cell into which it is intended to be delivered and the mechanism by which that effect is to be carried out. For example, the genetic material may be useful in gene therapy, for example in order to express a desired gene in a cell or group of cells. Such genetic material is typically in the form of plasmid DNA or viral vector encoding the desired gene and operatively linked to appropriate regulatory sequences such as promoters, enhancers and the like such that the plasmid DNA is expressed once it has been delivered to the cells to be treated. Examples of diseases susceptible to gene therapy include haemophilia B (Factor IX), cystic fibrosis (CTFR) and spinal muscular atrophy (SMN-1).

Genetic material can also be used for example in immunisation to express one or more antigens against which it is desired to produce an immune response. Thus, the nucleic acid to be loaded into the EV can encode one or more antigens against which is desired to produce an immune response, including but not limited to tumour antigens, antigens from pathogens such as viral, bacterial or fungal pathogens.

Genetic material can also be used in gene silencing. Such gene silencing may be useful in therapy to switch off aberrant gene expression or in animal model studies to create single or more genetic knock outs. Typically such genetic material is provided in the form of siRNAs. For example, RNAi molecules including siRNAs can be used to knock down DMPK with multiple CUG repeats in muscle cells for treatment of myotonic dystrophy. In other examples, plasmids expressing shRNA that reduces the mutant Huntington gene (htt) responsible for Huntington's disease can be delivered with neuron specific exosomes. Other target genes include BACE-1 for the treatment of Alzheimer's disease. Some cancer genes may also be targeted with siRNA or shRNAs, such as ras, c-myc and VEGFR-2. Brain targeted siRNA loaded exosomes may be particularly useful in the silencing of BACE-1 in Alzheimer's disease, silencing of alpha-synuclein in Parkinson's disease, silencing of htt in Huntingdon's disease and silencing of neuronal caspase-3 used in the treatment of stroke to reduce ischaemic damage.

Antisense modified oligonucleotides including 2′-O-Me compounds and PNA can be used. For example, such oligonucleotides can be designed to induce exon-skipping for example the mutant dystrophin gene can be delivered to muscle cells for the treatment of Duchenne Muscular Dystrophy, antisense oligonucleotides which inhibit hairpin loops, for example in the treatment of myotonic dystrophy and trans-splicing oligonucleotides, for example for the treatment of spinal muscular atrophy.

Release Systems

Two types of release systems, in particular, are envisaged for use with the present invention. The first release system may be a release system that can be activated to release the second protein and/or cargo from the EV, in particular when the second protein and/or cargo is present on the outer surface of the EV. The second release system may facilitate the release of an EV from an endosome, such as from a late endosome. However, as will be apparent to the skilled person, the presence of either release system may not be essential, for example when the therapeutic effect of an EV arises from the an interaction between cargo on the surface of an EV interacting with a moiety on the outer surface of a cell.

The first release system may be an organic compound-based or polypeptide-based release system. Typically, the organic compound or polypeptide of this release system will be a linker forming a covalent link between: (a) the PS-binding protein and/or the EV-binding protein, and the second protein; (b) the PS-binding protein and/or the EV-binding protein, and the cargo; and/or (c) the second protein and the cargo. The linker may be activated to split into at least two discrete units, wherein the discrete units of the linker are not attached to each other. Thus, when the linker is activated, the second protein and/or cargo is released from the lipid bilayer of the EV. If the second protein/and or cargo is attached to the outer surface of the EV, then activating the linker results in the second protein and/or cargo being released from its association with the EV. Suitable activatable linkers will be apparent to the skilled person from the prior art. For example, a linker may be activated a specific wavelength or light, or due to a change in pH such as due to the acidification of endosomes.

When the release system is a polypeptide-based system it may be selected from the group comprising various releasable polypeptide interaction systems which may be activated or triggered without the need for exogenous stimuli (i.e. the release systems are typically triggered by endogenous activity within a cell or an EV, or essentially within any biological system), for instance a cis-cleaving polypeptide-based release system (e.g. based on inteins), a nuclear localization signal (NLS)—NLS binding protein (NLSBP)-based release system or release systems based on other protein domains. In one embodiment, a monomeric light-induced cleavage-based release system may be utilized, where only a very short boost of light is utilized to start an endogenous proteolytic cleavage of a monomeric protein domain and release the Pol.

The second release system may facilitate the release of an EV that has been taken up into the cell, for example by endocytosis. Endocytosis describes the physiological uptake of extracellular materials by cells through their encapsulation in vesicular compartments termed endosomes. Thus, when taken up into cells by endocytosis, an EV may be encapsulated in an endosome and it may be desirable to facilitate the release of an EV from an endosome. The skilled person would recognize that a number of approaches could be combined with the present invention to facilitate release of an EV from an endosome, such as a molecule that enhances release of an EV from an endosome. Such a molecule may be co-administered with the EV. Alternatively, or in addition, the EV may be modified to comprise such a molecule, for example, where the molecule that enhances release of an EV from an endosome is linked to:
(a) an EV membrane-bound moiety, optionally wherein the membrane-bound moiety is cholesterol, or a protein and/or peptide; and/or
(b) a PS-binding protein, such as a PS-binding protein according to the present invention.

The molecule that enhances release of an EV from an endosome may be a pH-sensitive membrane-perturbing molecule. The molecule that enhances release of an EV from an endosome may be a molecule that binds to protons such as chloroquine, or variants thereof such as hydroxychloroquine. In other words, the molecule that binds to protons may be any suitable endosomolytic molecule.

Other endosomal escape peptides may be used in combination with the present invention, such as one or more endosomal escape peptide selected from HIV TAT PDT (peptide/protein transduction domain), KALA, GALA and INF-7 (derived from the N-terminal domain of influenza virus hemagglutinin HA-2 subunit), endosomal escape moieties that act by causing membrane fusion such as Diphtheria toxin T domain, proton sponge type endosomal escape moieties such as lipids with histidine or imidazole moieties and cell penetrating peptides (CPPs) and other moieties that enable endosomal escape by acting to puncture membranes. CPPs are typically less than 50 amino acids but may also be longer, are typically highly cationic and rich in arginine and/or lysine amino acids and have the ability to gain access to the interior of virtually any cell type, exemplary CPPs may be transportan, transportan 10, penetratin, MTS, VP22, CADY peptides, MAP, KALA, PpTG20, proline-rich peptides, MPG peptides, PepFect peptides, Pep-1, L-oligomers, calcitoninpeptides, arginine-rich CPPs such as poly-Arg, tat and combinations thereof).

Targeting

The EV of the present invention may be targeted to a desired cell type or tissue. This targeting is achieved by expressing on the surface of the EV of a targeting moiety which binds to a cell surface moiety expressed on the surface of the cell to be targeted. Typically the targeting moiety is a peptide which may be expressed as a fusion protein with a transmembrane protein typically expressed on the surface of the EV.

In more detail, the EV of the invention can be targeted to particular cell types or tissues by expressing on their surface a targeting moiety such as a peptide. Suitable peptides are those which bind to cell surface moieties such as receptors or their ligands found on the cell surface of the cell to be targeted. Examples of suitable targeting moieties are short peptides, scFv and complete proteins, so long as the targeting moiety can be expressed on the surface of the EV and does not interfere with cargo carrying capacity of the EV and PS-binding activity. Typically the targeting peptide is heterologous to the transmembrane EV protein. Peptide targeting moieties may typically be less than 100 amino acids in length, for example less than 50 amino acids in length, less than 30 amino acids in length, to a minimum length of 10, 5 or 3 amino acids.

Targeting moieties can be selected to target particular tissue types such as muscle, brain, liver, pancreas and lung for example, or to target a diseased tissue such as a tumour. In a particularly preferred embodiment of the present invention, the EV are targeted to brain tissue.

Specific examples of targeting moieties include muscle specific peptide, discovered by phage display, to target skeletal muscle, a 29 amino acid fragment of Rabies virus glycoprotein that binds to the acetylcholine receptor or a fragment of neural growth factor that targets its receptor to target neurons, the secretin peptide that binds to the secretin receptor can be used to target biliary and pancreatic epithelia. As an alternative, immunoglobulins and their derivatives, including scFv antibody fragments can also be expressed as a fusion protein to target specific antigens. As an alternative, natural ligands for receptors can be expressed as fusion proteins to confer specificity, such as NGF which binds NGFR and confers neuron-specific targeting. The peptide targeting moiety may be expressed on the surface of the EV by expressing it as a fusion protein with an EV transmembrane protein. A number of proteins are known to be associated with EVs; that is they are incorporated into the EV as it is formed. The preferred proteins for use in targeting the EVs of the present invention are those which are transmembrane proteins.

The EV proteins which is comprised in the fusion proteins as per the present invention may be selected from the group comprising the following non-limiting examples: CD9, CD53, CD63, CD81, CD54, CD50, FLOT1, FLOT2, CD49d, CD71, CD133, CD138, CD235a, ALIX, Syntenin-1, Syntenin-2, LAMP-2B, TSPAN8, syndecan-1, syndecan-2, syndecan-3, syndecan-4, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD11, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, ARRDC1, PDGFRN, ATP2B2, ATP2B3, ATP2B4, BSG, IGSF2, IGSF3, IGSF8, ITGB1, ATPlA2, ATP1A3, ATP1A4, ITGA4, SLC3A2, ATPlA1, ATP1B3, ATP2B1, LFA-1, LGALS3BP, Mac-1 alpha, Mac-1 beta, MFGE8, SLIT2, STX3, TCRA, TCRB, TCRD, TCRG, VTI1A, VTI1B, and any other EV proteins, and any combinations, derivatives, domains, variants, mutants, or regions thereof. Particularly advantageous EV proteins include CD63, CD81, CD9, CD82, CD44, CD47, CD55, LAMP-2B, ICAMs, integrins, ARRDC1, syndecan, syntenin, and Alix, as well as derivatives, domains, variants, mutants, or regions thereof.

The EV of the present invention may be targeted to a desired cell type or tissue. For example, the EV of the present invention may be targeted to a cancer cell and/or the blood-brain-barrier (BBB). When present in the blood, EV of the present invention may cross the BBB. At least 0.01%, at least 0.1%, at least 1%, at least 2%, at least 5%, at least 10%, at least 20%, at least 25%, at least 30%, at least 40%, at least 50%, at least 60%, at least 70%, at least 80%, at least 90%, at least 95% or at least 99% of the total number of EVs delivered into a patient may be targeted to the desired cell type or tissue and/or cross the BBB.

Compositions and Therapeutic Applications

Compositions of the invention comprise at least an EV as set out in other sections of this disclosure. Thus, the compositions of the invention may be used for targeted delivery of cargo, such as RNA-based drugs. In addition, the compositions of the invention may be used as a research tool, a diagnostic tool, an imaging tool, biological reference material, an experimental control and/or an experimental standard.

Compositions comprising EVs loaded with cargo may be useful for vaccination, treating immune-privileged sites such as the eye, delivering cargo across the blood brain barrier, treating acute conditions such as severe combined immunodeficiency and treating chronic conditions such as myotonic dystrophy. The invention provides a simple, robust and efficient way to load cargo such as RNA-based drugs onto EVs to produce the compositions of the invention.

The use of EV compositions to deliver cargo offers a number of advantages over conventional means of delivering such cargo. For example, when cargo is delivered using EV compositions, it may be protected from degradation and may be more stable; cargo may be delivered to a target tissue, such as a specific type of cancer, more efficiently and/or more specifically than if not associated with an EV. In addition, the cargo may be less likely to elicit an immune response when contained within EVs as it is not freely available for detection by immune cells and/or binding to antibodies. Thus, an EV and/or EV composition may be substantially immunologically inert. An EV composition may be engineered to be substantially immunologically inter, for example by the introduction of a moiety such as a protein domain that masks the EV composition from the immune system. By way of example, an RNA binding domain attached to the G58 peptide of GAPDH protein may be used to mask RNA based drugs from activations of the innate immune response. Other potential advantages of the use of EVs to deliver cargo include avoiding drug resistance, such as the upregulation of drug transporters such as ABC-transporters, rapid tissue internalisation, single particle uptake, wide therapeutic index, broad biodistribution and good bioavailability.

The EV composition of the invention may be loaded with any cargo that has utility in the treatment and/or prevention of a condition, disease or disorder. The cargo to be loaded into the EV is chosen on the basis of the desired effect of that protein and/or peptide on the cell into which it is intended to be delivered and the mechanism by which that effect is to be carried out. A single cargo molecule may be incorporated into the EV. Alternatively, more than one cargo molecule may be incorporated into the EV. The more than one cargos may act on the same or different targets to bring about their therapeutic and/or preventative effect.

The cargo loaded into the composition may be a cargo that is not used to generate an immune response. The cargo may be selected to provide a therapeutic benefit itself, and is not intended to be used to generate an immune response against the cargo. The cargo to be incorporated into the EV composition may be useful, for example, in the prophylaxis and/or treatment and/or alleviation of a variety of diseases, typically via the delivery of essentially any type of drug cargo, such as for instance mRNA, antisense or splice-switching oligonucleotides, siRNA, pDNA, peptides, proteins, antibodies, antibody-drug conjugates, gene editing technology such as CRISPR-Cas9, TALENs, meganucleases, or vesicle-based cargos such as viruses (e.g. AAVs, lentiviruses, etc.) or EVs (exosomes, microvesicles and the like). Non-limiting examples of diseases and conditions that are suitable targets for treatment using the peptide delivery system described herein include the following non-limiting examples: Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumour necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying aetiology, graft-vs-host disease, Duchenne muscular dystrophy and other muscular dystrophies, In-born errors of metabolism including: Disorders of carbohydrate metabolism e.g., G6PD deficiency galactosemia, hereditary fructose intolerance, fructose 1,6-diphosphatase deficiency and the glycogen storage diseases, Disorders of organic acid metabolism (organic acidurias) such as alkaptonuria, 2-hydroxyglutaric acidurias, methylmalonic or propionic acidemia, multiple carboxylase deficiency, Disorders of amino acid metabolism such as phenylketonuria, maple syrup urine disease, glutaric acidemia type 1, Aminoacidopathies e.g., hereditary tyrosinemia, nonketotic hyperglycinemia, and homocystinuria, Hereditary tyrosinemia, Fanconi syndrome, Primary Lactic Acidoses e.g., pyruvate dehydrogenase, pyruvate carboxylase and cytochrome oxidase deficiencies, Disorders of fatty acid oxidation and mitochondrial metabolism such as short, medium, and long-chain acyl-CoA dehydrogenase deficiencies also known as Beta-oxidation defects, Reye's syndrome, Medium-chain acyl-coenzyme A dehydrogenase deficiency (MCADD), MELAS, MERFF, pyruvate dehydrogenase deficiency, Disorders of porphyrin metabolism such as acute intermittent porphyria, Disorders of purine or pyrimidine metabolism such as Lesch-Nyhan syndrome, Disorders of steroid metabolism such as lipoid congenital adrenal hyperplasia, congenital adrenal hyperplasia, Disorders of mitochondrial function such as Kearns-Sayre syndrome, Disorders of peroxisomal function such as Zellweger syndrome and neonatal adrenoleukodystrophy, congenital adrenal hyperplasia or SmithLemli-Opitz, Menkes syndrome, neonatal hemochromatosis, Urea cycle disorders such as N-Acetylglutamate synthase deficiency, carbamoyl phosphate synthetase deficiency, ornithine transcarbamoylase deficiency, citrullinemia (deficiency of argininosuccinic acid synthase), argininosuccinic aciduria (deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1, orotic aciduria (deficiency in the enzyme uridine monophosphate synthase UMPS), all of the lysosomal storage diseases, for instance Alpha-mannosidosis, Betamannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Mucopolysaccharidosis, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease, etc. cystic fibrosis, primary ciliary dyskinesia, pulmonary alveolar proteinosis, ARC syndrome, Ret syndrome, neurodegenerative diseases including Alzheimer's disease, Parkinson's disease, GBA associated Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumour, Brainstem glioma, Brain cancer, Brain tumour (cerebellar astrocytoma, cerebral astrocytoma/malignant glioma, ependymoma, medulloblastoma, supratentorial primitive neuroectodermal tumours, visual pathway and hypothalamic glioma), Breast cancer, Bronchial adenomas/carcinoids, Burkitt's lymphoma, Carcinoid tumour (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumour, Endometrial cancer, Ependymoma, Esophageal cancer, Extracranial germ cell tumour, Extragonadal Germ cell tumour, Extrahepatic bile duct cancer, Eye Cancer (Intraocular melanoma, Retinoblastoma), Gallbladder cancer, Gastric (Stomach) cancer, Gastrointestinal Carcinoid Tumour, Gastrointestinal stromal tumour (GIST), Germ cell tumour (extracranial, extragonadal, or ovarian), Gestational trophoblastic tumour, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumour), Ovarian germ cell tumour, Ovarian low malignant potential tumour, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumours sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumour, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilms' tumour.

Thus, provided is a composition comprising an EV according to the present invention and at least one pharmaceutically acceptable excipient, for use in a method of therapy in a subject. Also provided is a composition comprising an EV according to the present invention and at least one pharmaceutically acceptable excipient, for use in a method of treating and/or preventing at least one of the therapeutic indications set out above.

Also provided is the use of a composition comprising an EV according to the present invention and at least one pharmaceutically acceptable excipient for the manufacture of a medicament for therapy. Also provided is the use of a composition comprising an EV according to the present invention and at least one pharmaceutically acceptable excipient, for the manufacture of a medicament for treating and/or preventing at least one of the therapeutic indications set out above.

Also provided is a method of treatment comprising providing an EV according to the present invention and at least one pharmaceutically acceptable excipient to a patient in need thereof. Also provided is a method of treatment and/or prevention comprising providing an EV according to the present invention and at least one pharmaceutically acceptable excipient to a patient in need thereof, wherein at least one of the therapeutic indications set out above is treated and/or prevented.

Delivery/Administration

The EVs of the invention may be administered by any suitable means. Administration to a human or animal subject may be selected from parenteral, intramuscular, intracerebral, intravascular (including intravenous), subcutaneous, intranasal, intracardiac, intracerebroventricular, intraperitoneal or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for any suitable means of administration, including parenteral, intramuscular, intracerebral, intravascular (including intravenous), intracardiac, intracerebroventricular, intraperitoneal, subcutaneous, intranasal or transdermal administration. Compositions for parenteral administration may include sterile aqueous solutions which may also contain buffers, diluents and other suitable additives. The EVs of the invention may be formulated in a pharmaceutical composition, which may include pharmaceutically acceptable carriers, thickeners, diluents, buffers, preservatives, and other pharmaceutically acceptable carriers or excipients and the like in addition to the EVs.

A “pharmaceutically acceptable carrier” (excipient) is a pharmaceutically acceptable solvent, suspending agent or any other pharmacologically inert vehicle for delivering one or more nucleic acids to a subject. Typical pharmaceutically acceptable carriers include, but are not limited to, binding agents (e.g. pregelatinised maize starch, polyvinylpyrrolidone or hydroxypropyl methylcellulose, etc); fillers (e.g. lactose and other sugars, microcrystalline cellulose, pectin, gelatin, calcium sulfate, ethyl cellulose, polyacrylates or calcium hydrogen phosphate, etc); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc); or wetting agents (e.g. sodium lauryl sulphate, etc).

The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavouring agents, preservatives, antioxidants, opacifiers, thickening agents and stabilizers. However, such materials, when added, should not unduly interfere with the biological activities of the components of the compositions provided herein.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual EVs, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific EV, the age, weight and condition of the subject to be treated, the severity of the disease and the frequency and route of administration. Different dosages of the EV may be administered depending on whether administration is by intramuscular injection or systemic (intravenous or subcutaneous) injection. Preferably, the dose of a single intramuscular injection is in the range of about 5 to 20 μg. Preferably, the dose of single or multiple systemic injections is in the range of 10 to 100 mg/kg of body weight.

It will be clear to the skilled artisan that when describing medical and scientific uses and applications of the EVs, the present invention normally relates to a plurality of EVs, i.e. a population of EVs which may comprise thousands, millions, billions or even trillions of EVs. EVs may be present in concentrations such as about 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml), or any other number larger, smaller or anywhere in between. In the same vein, the term “population”, which may e.g. relate to an EV comprising a certain cargo shall be understood to encompass a plurality of entities constituting such a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of the cargo of interest, any targeting moieties present on the EVs, the pharmaceutical formulation, etc.

Due to EV clearance (and breakdown of any cargo molecule), the patient may have to be treated repeatedly, for example once or more daily, weekly, monthly or yearly. Persons of ordinary skill in the art can easily estimate repetition rates for dosing based on measured residence times and concentrations of the EV in bodily fluids or tissues. Following successful treatment, it may be desirable to have the patient undergo maintenance therapy, wherein the EV is administered in maintenance doses, ranging from 0.01 mg/kg to 100 mg per kg of body weight, once or more daily, to once every 20 years.

A composition of the invention may be co-administered with one or more other agent. The one or more other agent may be administered separately to the composition of the invention, at substantially the same time as the composition of the invention, or as a single composition comprising the EV of the invention in combination with the one or more other agent. Thus, combination therapy comprising the EV of the invention and one or more other agent is envisaged. The one or more other agent may be loaded into the EV, for example it may be encapsulated inside the EV or bound to the surface of the EV. By way of example, the composition of the invention may be co-administered with a cell-penetrating peptide (CCP) to assist intracellular delivery and/or cell-specific targeting.

Isolating EVs and Production of EV Compositions

Suitable cells for production of EVs will be apparent to the skilled person. Any EV-producing cell can be utilized. Suitable physiological fluids from which EVs can be isolated will also be apparent to the skilled person. EVs can be collected from a cell culture medium and/or a physiological fluid by any suitable method. EVs may be isolated from a suitable cell bank. Alternatively, EVs may be isolated form any autologous patient-derived, heterologous haplotype-matched or heterologous stem cells so to reduce or avoid the generation of an immune response in a patient to whom the EVs are delivered.

Typically a preparation of EVs can be prepared from cell culture tissue supernatant or physiological fluid by centrifugation, filtration or combinations of these methods. For example, EVs can be prepared by differential centrifugation, that is low speed (<20,000 g) centrifugation to pellet larger particles followed by high speed (>100,000 g) centrifugation to pellet EVs, size filtration with appropriate filters (for example, 0.22 μm filter), gradient ultracentrifugation (for example, with sucrose gradient) or a combination of these methods. Isolated EVs may be further purified, concentrated and/or diluted as appropriate.

Isolated EVs may be further manipulated to produce the EVs comprised in the compositions of the invention, for example through the addition of additional molecules. Alternatively, the isolated EV may already represent the EVs comprised in the compositions of the invention, for example due to the endogenous expression in the host cell of the PS-binding protein and/or PS-binding protein fused to a second protein, and the EV cargo. In other words, EVs may also be loaded by transforming or transfecting a host cell with a nucleic acid construct which expresses therapeutic cargo of interest, such that the therapeutic cargo binds directly or indirectly to the PS-binding protein and is hence loaded into the EV as the EV are produced from the cell.

The EV and/or EV composition of the invention may be produced by providing an isolated EV expressing PS and/or another lipid molecule that is not PS. In one embodiment, the EV may express PS and at least one other lipid molecule that is not PS. The presence of a lipid molecule that is not PS may facilitate and/or enhance binding of the PS-binding protein to the EV. The lipid molecule such as PS may be expressed on the outer surface of the EV, the inner surface of the EV, or both the out and inner surfaces of the EV. The isolated PS-expressing and/or lipid-expressing EV may then be accordingly brought into contact with a PS-binding and/or lipid-binning peptide to allow the PS-binding or lipid-binding peptide to bind to the PS and/or lipid, thereby producing an EV composition comprising a PS-binding peptide and/or lipid-binding peptide bound to the surface of the EV by means of an interaction between the phosphatidylserine-binding peptide and/or lipid-binding peptide and PS and/or lipid on the surface of the EV. The PS-binding and/or lipid-binding peptide may be present on the outer surface and/or the inner surface of the EV.

The PS-binding and/or lipid-binding peptide may be linked to a second protein. Linkage may occur chemically after the PS-binding or lipid-binding peptide has bound to the EV. Alternatively, linkage of the PS-binding peptide and/or lipid-binding peptide, and the second protein, may occur chemically and/or recombinantly before the PS-binding peptide and/or lipid-binding peptide is brought into contact with the EV. Alternatively, linkage of the PS-binding peptide and/or lipid-binding peptide, and second protein, may occur recombinantly in the host cell. The second protein, peptide and/or small molecule drug may be selected from one or more of an enzyme, an antibody and/or antigen-binding variant or fragment thereof, a single chain variable fragment (scFv) and a cargo-binding protein. The cargo-binding protein and/or peptide may be a RNA- and/or DNA-binding protein, for example a protein and/or peptide selected from one or more of TRBP2 and PKdsRBD2 and/or a RNA- and/or DNA-binding variant or fragment of anyone thereof.

The EV comprising a PS-binding protein and/or lipid-binding protein, or a PS-binding protein and/or lipid-binding protein, linked to a second protein, may be contacted with a cargo, such that the cargo binds to the PS-binding protein and/or lipid-binding protein, or to the second protein. The cargo may be selected from one or more of a protein, a peptide, an antibody and/or antigen binding variant or fragment thereof, a single chain variable fragment (scFv), a nucleic acid, a nucleic acid analogue, gRNA, miRNA, shRNA, siRNA, piRNA, PMO and DNA.

Thus, a further advantage of the present invention is that the EV composition may be produced with minimal steps. For example, no further substantive processing of the EV may be necessary following isolation of the EV from a host cell, for example if the host cell produces the cargo and a PS-binding protein-cargo binding protein fused to a cargo-binding protein. Alternatively, it may be possible to load an isolated EV with cargo in a single step, for example by mixing an isolated EV with cargo and a PS-binding protein-cargo binding protein fused to a cargo-binding protein.

### EXAMPLES

The invention is described in more detail below with reference to the following non-limiting Examples, which are intended to aid the comprehension of the invention.

### Example 1—Isolation, Purification and Characterisation of Extracellular Vesicles

Standard protocols for extracellular vesicles (EVs) isolation were followed. Briefly, HEK293T, MSCs, HeLa, SKOV-3 and B16 melanoma cells were seeded (10 million cells seeded in each plate) into 150 cm² tissue culture plates (Star Labs), using DMEM+10 FBS media (Thermo Scientific). For MSCs cells, RPMI+10% FBS media was used for culturing and seeding of the cells. After 24 h, DMEM+10 FBS media was replaced with reduced optiMEM media (Thermo Scientific). Cells were further incubated in optiMEM media for 48 h followed by collection of the media from the cells for isolation of extracellular vesicles. To remove dead and floating cells, the media was centrifuged at 500×g for 5 min. The media was gently transferred into fresh tubes and centrifuged at 3000×g for 20 min at 4° C. to pellet down cell fragments and remaining cell debris. Purified media was concentrated by tangential ultrafiltration (TFF), using 100 kDa cut-off membrane (Sartorius UK limited). With TFF, the final volume of the media was reduced to 10 ml, which was aliquoted into 1 mL tubes and centrifuged at 10,000×g to remove bigger particles such as apoptotic vesicles. Finally, the media was concentrated to 2 mL by centrifugal spin filters (100 kDa cut-off size, Millipore), and loaded sepharose-4 fast flow gel-filtration column (GE Healthcare) to purify extracellular vesicles from proteins and nucleic acid of the media. Isolated EVs were characterised for size and density by Nanosight (Malvern Analytical), using nanoparticle tracking analysis software. EV markers, specifically exosomal makers, such as CD63, CD81, Alix and SGT101 were detected by immunoblotting using monoclonal antibodies (Abcam) and chemiluminescent detection system.

### Example 2—Cloning, Expression and Purification of Glyceraldehyde 3-Phosphate Dehydrogenase (GAPDH) Protein from BL21 (DE3) E. coli Cells

GAPDH protein fused to Flag tag at C-terminus and (His)6 tag at N-terminus was cloned into pET-28b(+) vector (Novagen). BL21(DE3) competent (E. coli cells) were used to express the protein. The cells were grown in bacterial shaking incubator at 37° C. till OD600 reached between 0.5 to 0.6. At this point, 1 mM Isopropyl β-D-1-thiogalactopyranoside (IPTG, Sigma) was added to culture to induce expression of the protein. After 4 h, cells were pelleted down and stored at −30° C. For purification of GAPDH protein, the pellet was resuspended in ice-cold sodium-phosphate buffer, pH8 (50 mM Na2HPO4/NaH2PO4, 10 mM Tris-Cl, 300 mM NaCl, 5 mM imidazole) and lysed by adding 100 ul of 50 mg/mL lysozyme (Sigma). After 20 min of incubation, bacterial cell lysate was sonicated (probe sonicator, Branson sonifier) and centrifuged at 20,000 G for 20 min (Beckman Centrifuge). Supernatant was collected and incubated with Ni-NTA matrix (Qiagen) in Sodium-Phosphate buffer, pH8.0 for 1 h at 4° C. After incubation, the mixture was centrifuged at 700×g for 5 min to pellet down Ni-NTA resin. The supernatant was transferred into fresh 50 mL tube for SDS-PAGE to analyse binding efficiency of the protein to Ni-NTA resin. Pelleted Ni-NTA resin was washed in sodium phosphate buffer, containing 10 mM imidazole. The protein was eluted in 250 mM Imidazole sodium phosphate buffer, pH 6.0. Purified GAPDH protein was analysed by SDS-PAGE. To remove Imidazole and other protein contaminants, purified GAPDH protein was passed through saphacryl S 200HR column (GE Health Care). Size of the protein was confirmed by mass spectrometry using MALDI.

Proteins such as G58T, G150T, GAPDH-TRBP were purified under denaturing conditions. For lysis and incubation of cell lysate with Ni-NTA resin, 6M urea was added to sodium phosphate buffer, pH8.0. During washing steps, 4M urea was added to Sodium Phosphate buffer containing 10 mM imidazole. During the elution step, 250 mM imidazole and 2M urea was added to sodium phosphate buffer, pH 6.0. Purified protein was refolded by diluting urea concentration to 0.5M using phosphate saline buffer (PBS). To remove aggregates of the protein, refolded solution of the protein was centrifuged at 30,000×g for 30 min. The proteins were concentrated by using centrifugal spin filters of 10 kDa molecular cut of size (Millipore). G58TF, G58TtF, G58PF, G58PtF were expressed in BL21 (DE3) Rosetta cells and purified by using denaturing condition as mentioned above.

### Example 3—Binding of GAPDH and its Derivatives to Extracellular Vesicles; and Binding and Update of siRNA by GAPDH Modified EVs

For Exogenous binding of GAPDH to EVs, purified EVs from HEK293T cells were concentrated to almost 2×10¹² EVs/ml. Increasing concentration of the protein was added to EVs (1×10¹² EVs) and incubated at 4° C. for 2 h. Excess of non-bound GAPDH was removed by gel filtration chromatography (sepharose 4 fast flow, GE Healthcare). Binding of GAPDH to EV surface was determined by western blotting using chemiluminescence. Binding of the protein was also confirmed by analysing the absorbance of EVs at 260 nm. Morphology and size distribution was determined by electron microscopy and NTA, using methods known in the art.

To determine the binding of siRNA to GAPDH-modified EVs, 20 pmole of siRNA was added to increasing molar concentration of EVs. The complexes were incubated for 5 min at room temperature. After the incubation, complexes were loaded into 2% agarose gel stained with 0.5 ug/ml ethidium bromide for visualization under UV illuminator. Binding of siRNA to EVs was determined by analysing the shift of siRNA on agarose gel. Free siRNA was used as a negative control for determining the binding. siRNA added to EVs remained in the wells, reflecting strong binding of siRNA to GAPDH-RNA binding proteins present on the surface of EVs.

For determining uptake of GAPDH-modified EVs in N2a cells, 20 pmoles of siRNA^{Cy-3} was loaded into the EVs. N2a cells seeded on coverslips were treated with the EVs for 8 h. After the incubation, cells were fixed in paraformaldehyde and nuclei of the cells was stained with Hoechst 33258 dye (Invitrogen). Fluorescence of siRNA taken up by the cells was visualized on Olympus FV1000 confocal microscope, using 63× objective lens. The data was assessed by using FV100 software supplied with the confocal microscope

### Example 4—Silencing of Gene in N2a Cells by GAPDH-Modified Extracellular Vesicles

For silencing of genes in N2a cells, different GAPDH fusion proteins were designed and expressed in bacterial cells. Second double-stranded RNA binding domain (dsRBD) of human TRBP protein (TAR-RNA binding protein) was fused at C-terminus of GAPDH proteins to mediate binding of siRNA to GAPDH protein. G58 peptide (region of GAPDH protein between 70 to 94 amino acid is designated as G58 peptide), which is responsible for binding to surface of EVs was also fused with dsRBD of TRBP protein to form G58-TRBP fusion protein for loading of siRNA into EVs. To enhance release of siRNA from late-endosomes, TAT and arginine rich peptide of flock house virus (FHV) were also attached to G58T protein to form different kinds of fusion proteins such as G58T (G58 peptide and dsRBD of TRBP), G58TF (G58 peptide, dsRBD of TRBP and FHV peptide) and G58T(tat)2 (G58 peptide, dsRBD of TRBP, two tat peptides). The proteins were purified from bacterial cells as mentioned previously. EVs from HEK293T cells were incubate with these proteins and excess of unbound protein was removed from EVs by gel-filtration chromatography. To optimise gene silencing in N2a cells, pre-designed GAPDH siRNA (Ambion) was loaded into G58 modified EVs. Different molar concentration of siRNA bound to G58-modified EVs were added to N2a cells. After 48 h of treatment, RNA from the cells were isolated by using Trizol method (Invitrogen). 250 ng of total RNA was used to synthesize cDNA using prime script reverse transcriptase kit (Takara). The cDNA was diluted with double-distilled water to 5 times, and 1 ul of it was used in real-time PCR, using gene specific probes and primers (tagman probes, Invitrogen). Amplification of beta-actin and hypoxanthine-guanine phosphoribosyl transferase (HPRT1) was used as an internal control. Quantification of GAPDH mRNA from N2a cells treated with scrambled-siRNA (scsiRNA) was used as a calibrator to determine the percentage of gene silencing. The data was analysed by ΔΔCt method using linear regression analysis software for calculation of PCR primer efficiency.

### Example 5—Silencing of Huntingtin Gene (HTT) Gene in Q140 Huntington Disease Model Animals

(a) Single Dose Regimen of EVs

Huntington's disease mouse model Q140 of 1 year age were used to assess silencing of mutant and wild HTT gene silencing by intravenous administration of siRNA-loaded EVs. In Q140 mouse model, the exon1 of mouse HTT is humanized and contains 140 CAG repeats. The mice have a slow progression of disease phenotype, which starts to appear at the age of 6 months. In this experiment, Q140 mice of 1 year age were grouped into Saline, Negative and treatment control groups. Each group contained 6 mice. Saline group was used as control to assess level of HTT mRNA after administering EVs loaded with either negative siRNA (Negative group) or a mixture of HTT siRNA (treatment group). RVG EVs bound to G58TF protein were used for HTT silencing experiment. EVs doses were calculated based on 0.5 mg/kg siRNA dosage regimen. Number of EVs needed to bind given amount of siRNA were calculated by gel-shift assay. 150-200 μl of EVs were administered intravenously. Second dose of EVs was given after 48 h of first dose. After 72 h of second dose, mice were euthanized and different sections of the brain were analyzed for HTT mRNA quantification using probe-based (Tagman probes, life technologies). Mice received saline was used as a calibrator to normalize levels of HTT mRNA in negative and treatment groups. Level of HTT mRNA in saline treated group was assigned as 1 and based on that the percentage of HTT silencing was determined by using ΔΔCt method. Multivariate ANOVA (2 tails) and post-hoc adjustment using Dunnett's test was used to calculate statistical significance between the means of the groups.

(b) Multiple Dose Regimen of EVs to Q140 Mice:

Q140 mice of 1 year in age were distributed randomly into three groups of 6 mice in each group. Mice were grouped into three groups with 6 mice in each group. Control group received saline. Negative group received EVs carrying negative siRNA. Treatment group received a mixture of siRNA (0.5 mg/kg dose) bound to EVs. In all HTT silencing experiments, G58TF bound to RVG EVs were used. A total of 4 doses were given to animals. Each dose was given regularly after 1 week of first dose. Post 72 h of last dose, Animals were euthanized and different sections of the brain were analyzed for HTT mRNA and protein level. Due to large size of HTT protein aggregates, we could not resolve the protein on western blots. Agarose gel electrophoresis for resolving aggregates (AGERA) were carried out to detect mutant HTT protein aggregates. However, we could not analyze the immunoblot due to high background noise. Immunohistochemistry of the cortex regions of the brain were carried out to determine level of mutant HTT protein aggregates and p62 inclusion bodies. Data was analyzed by GraphPad Prism software. Multivariate ANOVA (2 tails) and post-hoc adjustment using Dunnett's test was used to calculate statistical significance between the means of the groups

### Example 6—Expressing the N-Terminal Region of Lactoferrin (Lactoferrin N) on the Surface of EVs

In a set of experiments designed to assess the feasibility of expressing the N-terminal region of iron-binding protein lactoferrin (lactoferrin N) on the surface of EVs to promote cell internalization via interaction with the lactoferrin receptor, the inventors observed that lactoferrin N, cleaved from a LAMP (Lysosomal associated membrane protein) fusion protein, continued to associate with the outer EV surface. Previously, it was demonstrated that cells take up extracellular iron by secreting EVs carrying surface glyceraldehyde-3-phosphate dehydrogenase (GAPDH), which attaches to the iron-binding proteins lactoferrin and transferrin. The inventors confirmed the presence of GAPDH on the outside of EVs isolated from different cell sources using a protease digestion assay (FIGS. 4a and 4b). Enzyme kinetic assay indicated that GAPDH present on outer surface of EVs was enzymatically active. (FIG. 4c). Co-immunoprecipitation experiments of HEK293T cell lysates and EVs demonstrated that the lactoferrin N domain interacts with GAPDH in cells and on the surface of EVs (FIG. 4d). Furthermore, incubation of isolated EVs with purified lactoferrin N protein resulted in efficient binding of the protein to EV surface. However, incubation of a different domain of lactoferrin, the N1.1 domain which lacks the GAPDH binding motif, with purified EVs did not result in binding to the surface of EVs (FIGS. 2a and 5a). Taken together, these experiments confirmed the role for GAPDH in tethering lactoferrin N on the surface of EVs.

### Example 7—Enhancing the Loading of Lactoferrin N on EVs

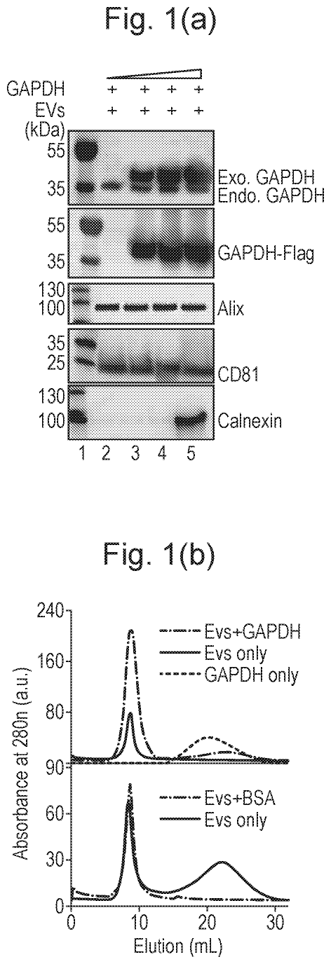

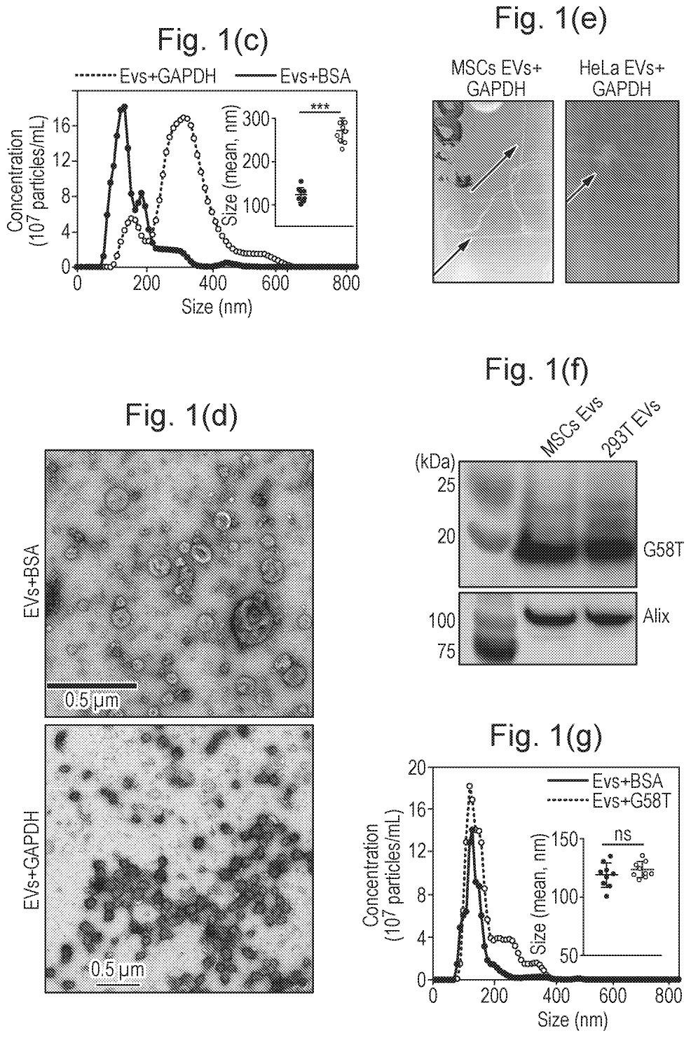

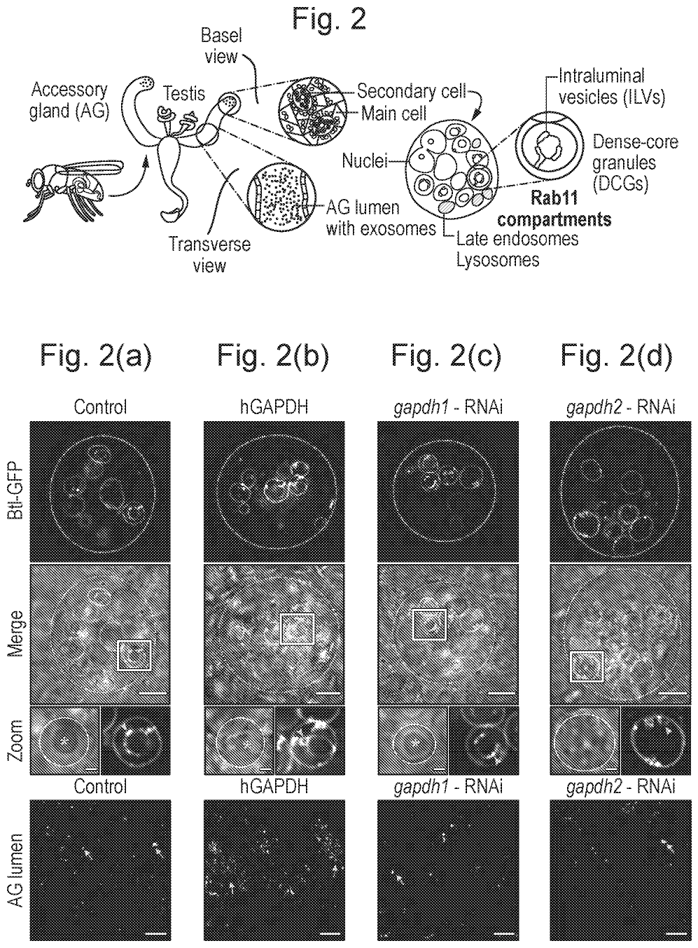

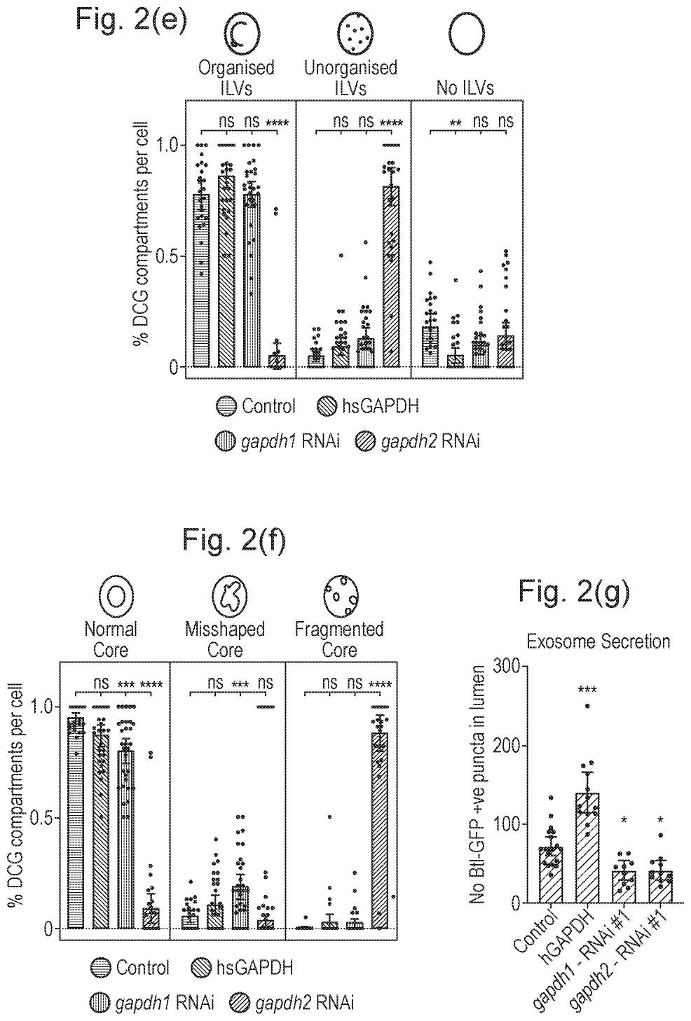

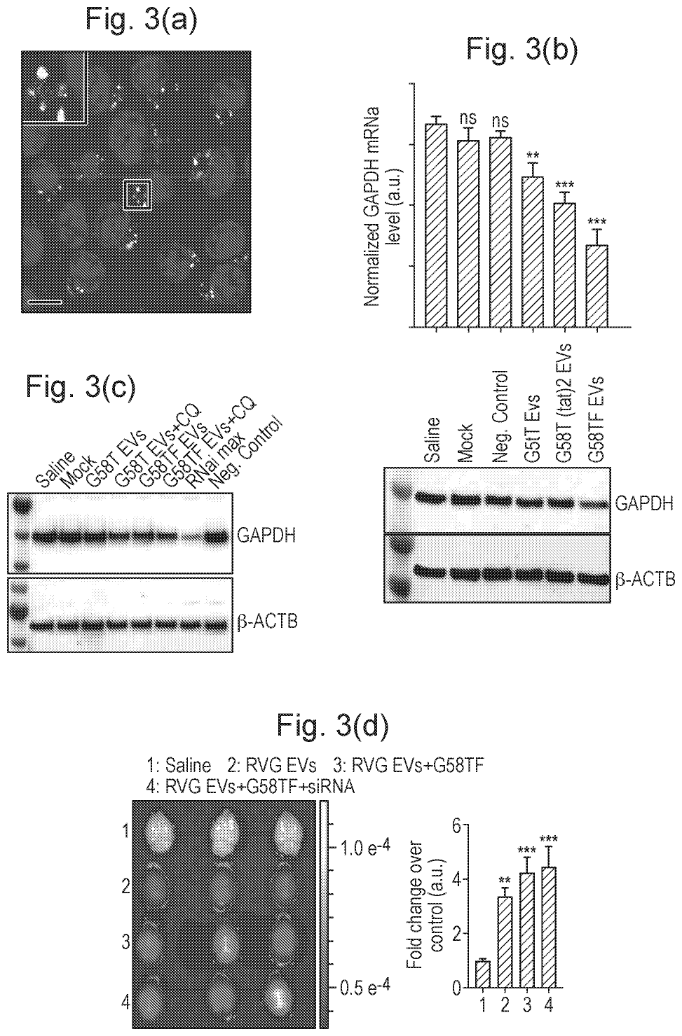

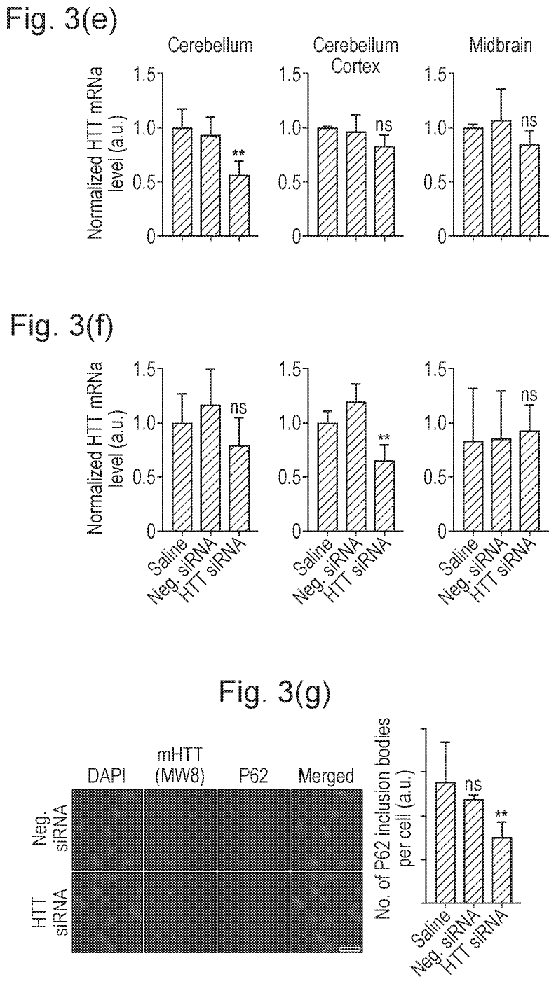

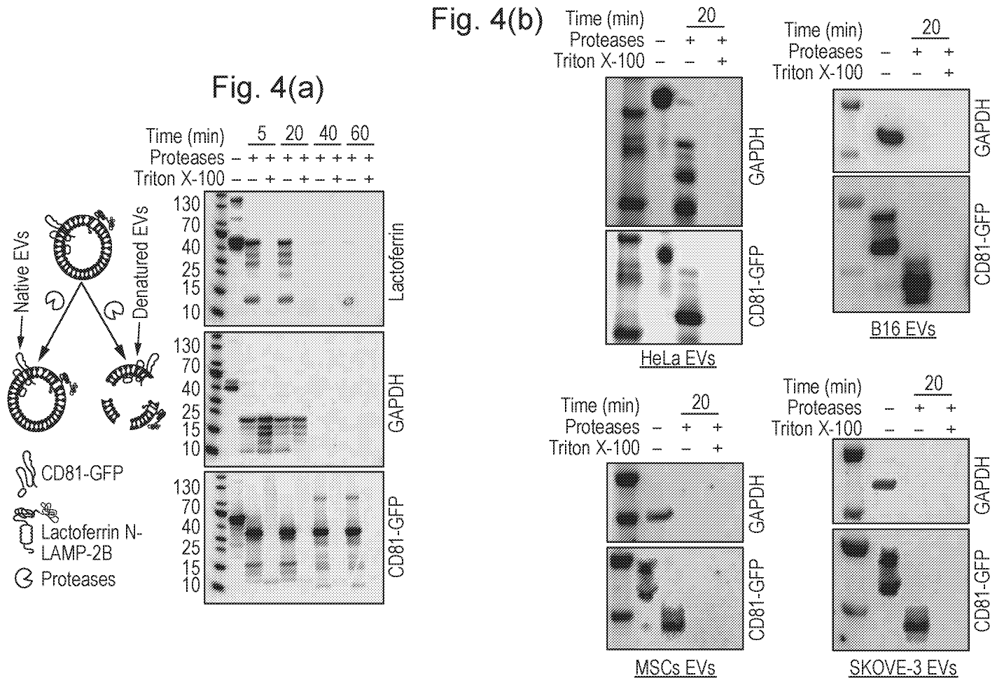

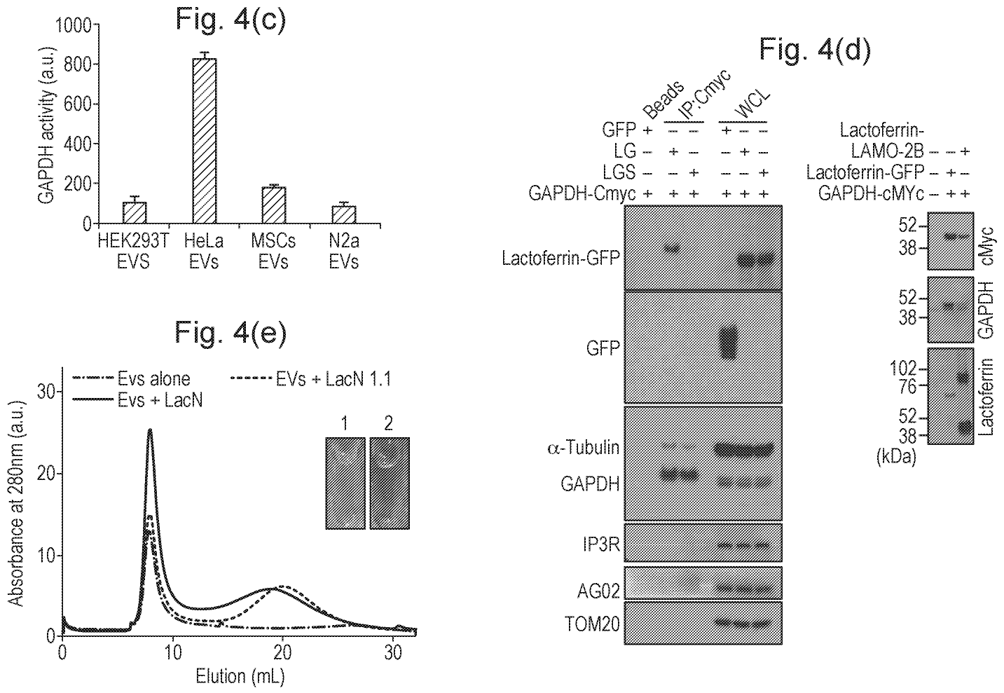

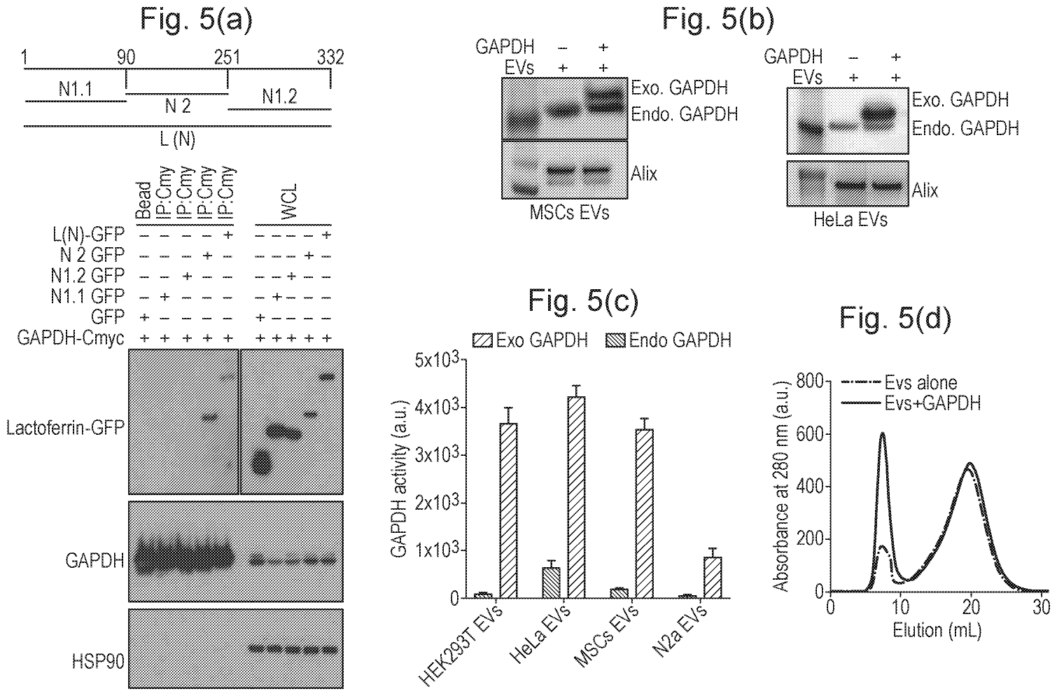

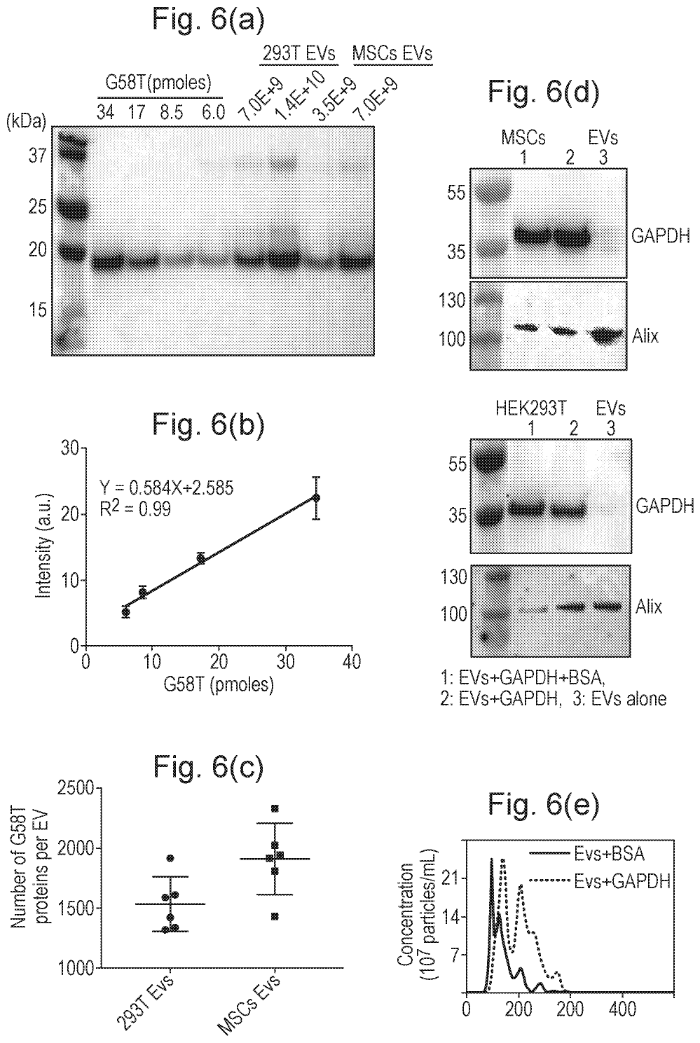

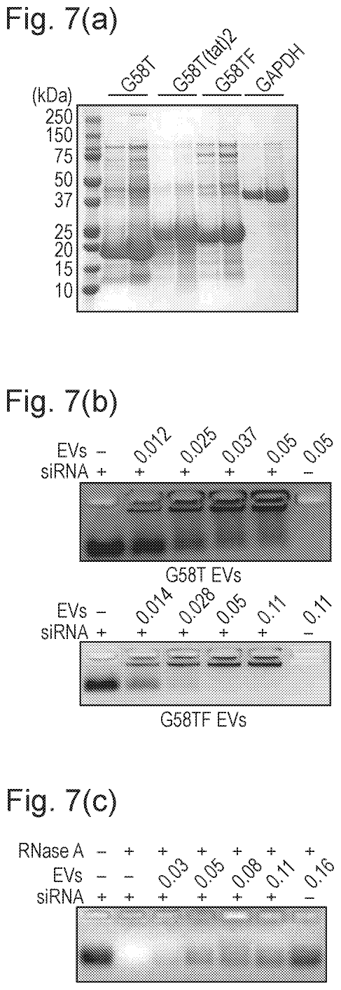

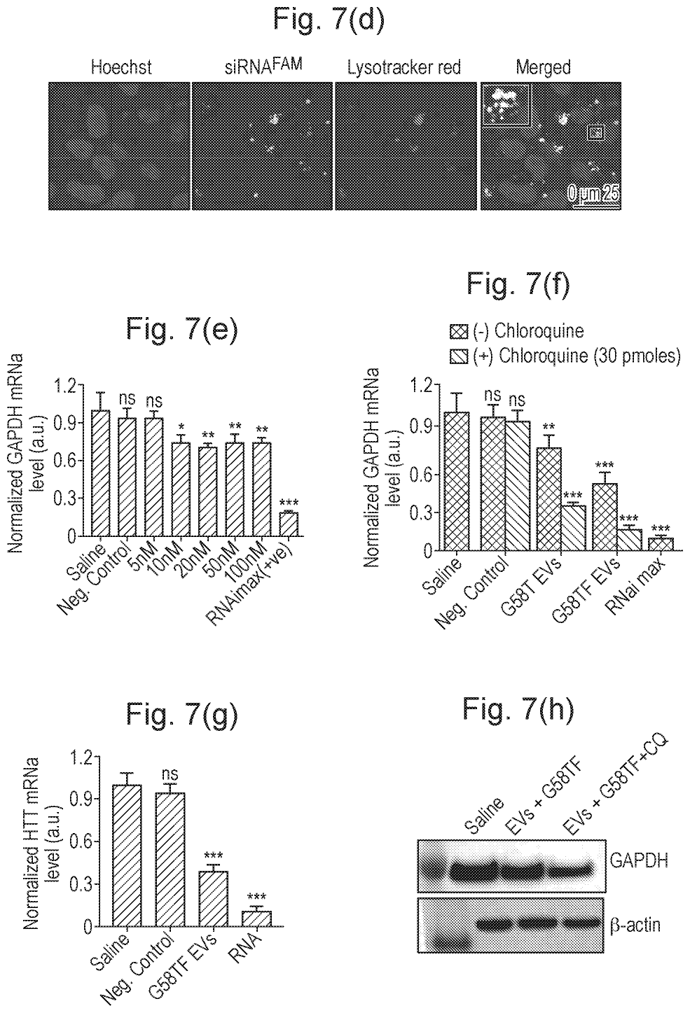

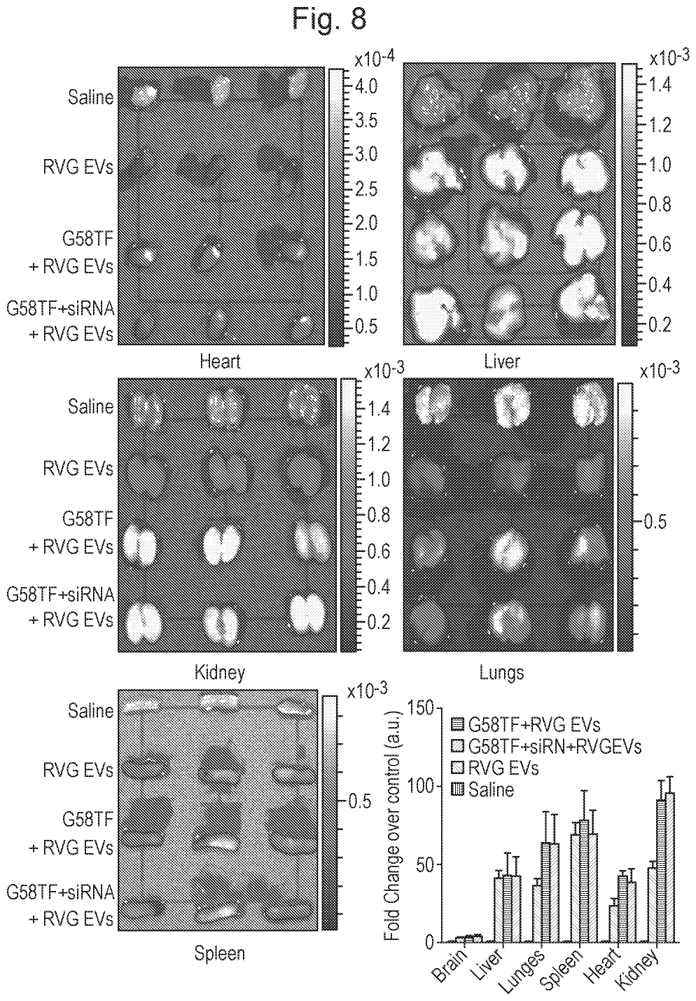

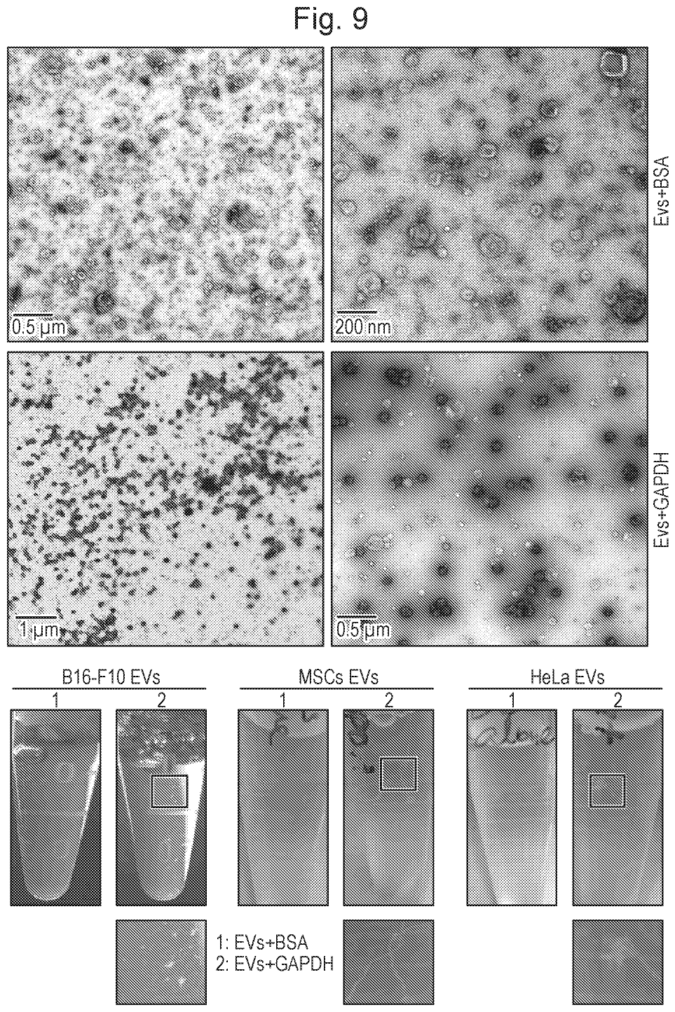

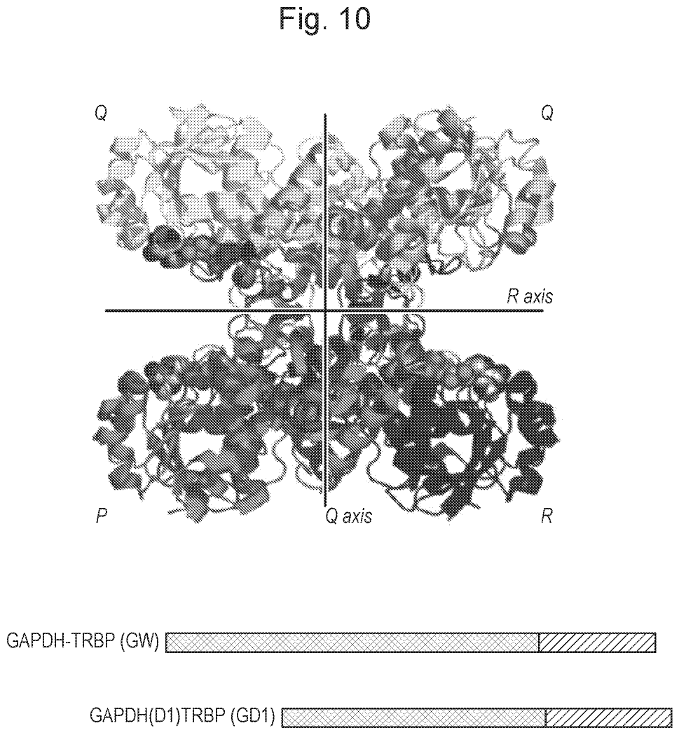

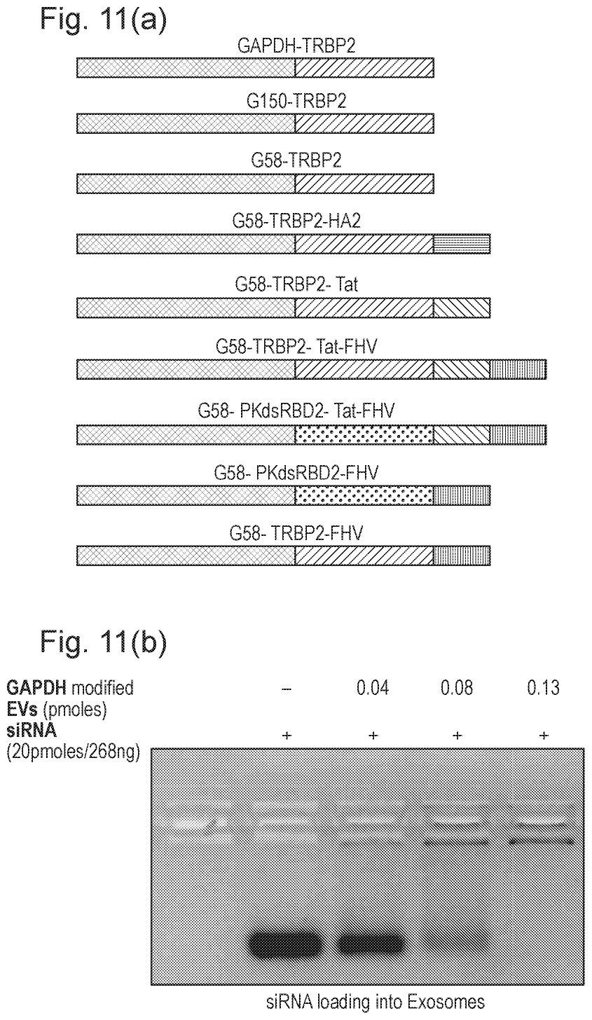

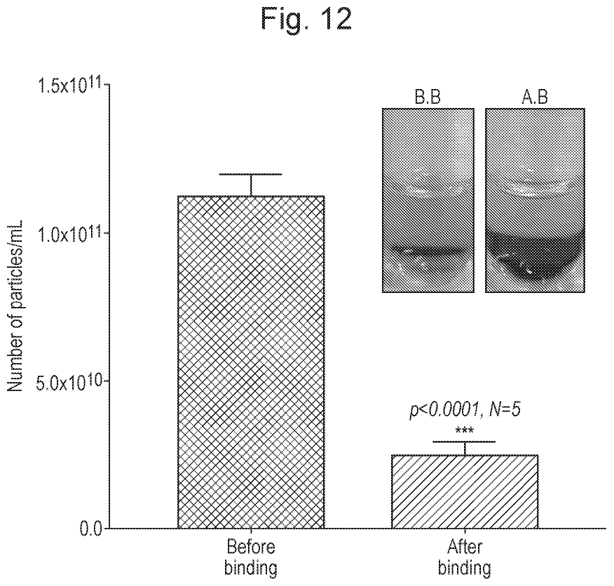

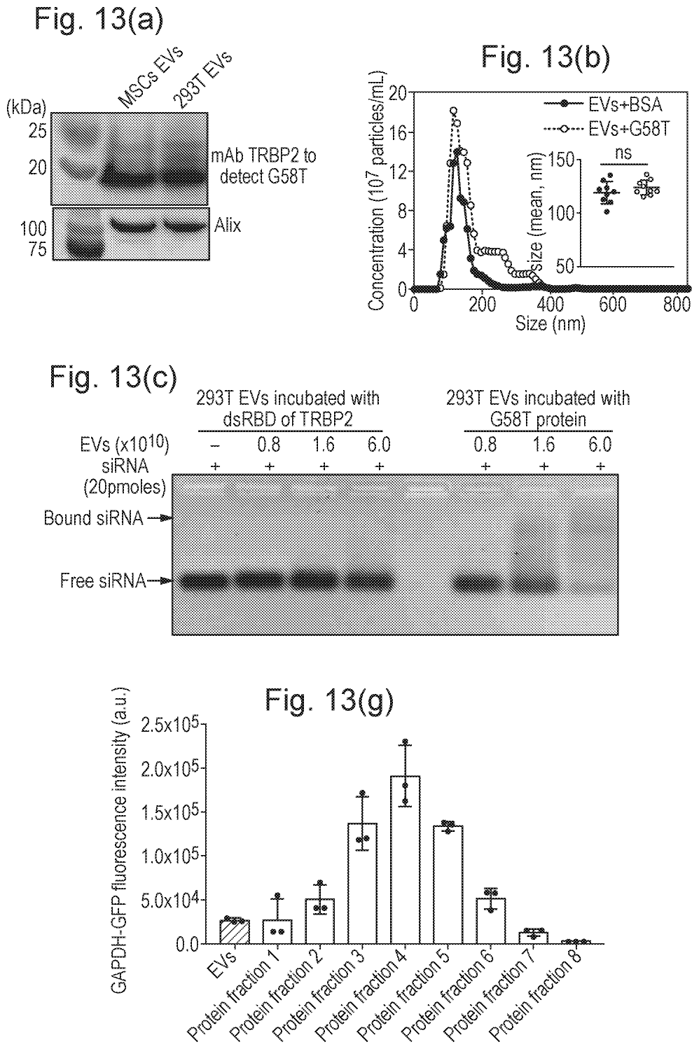

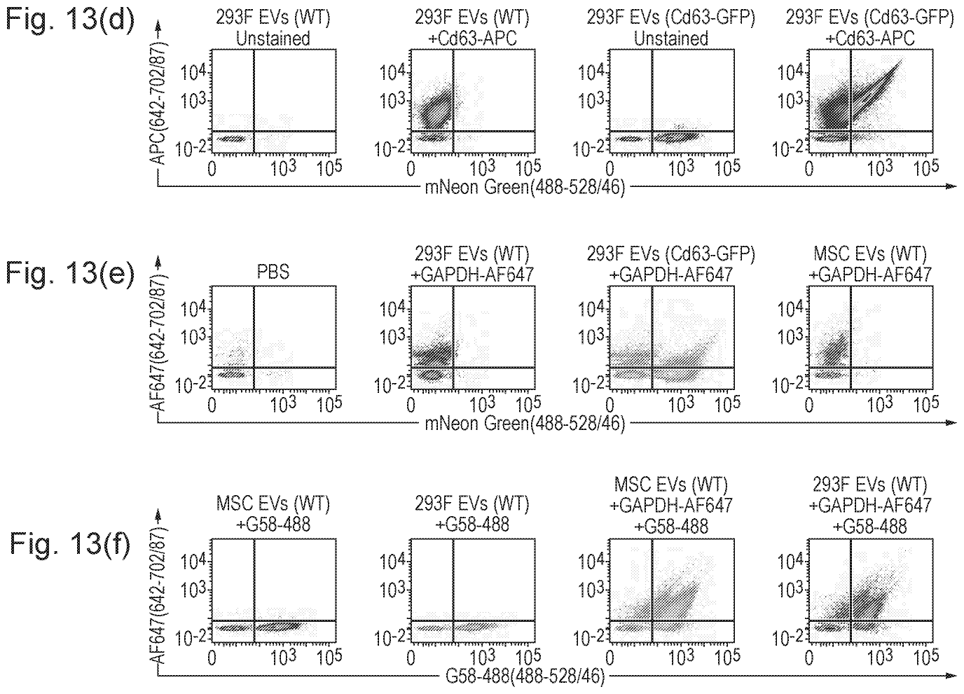

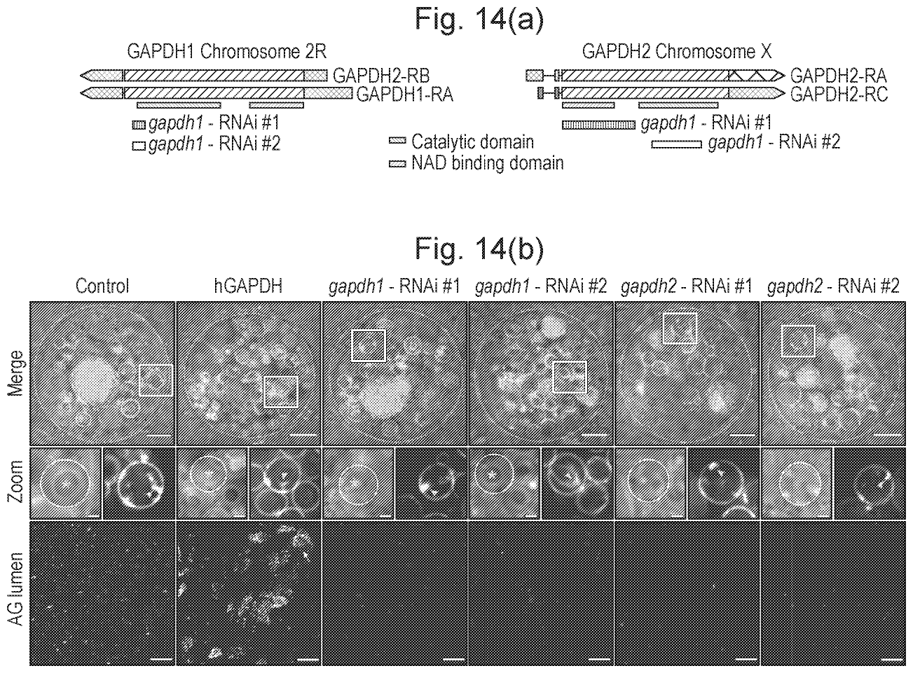

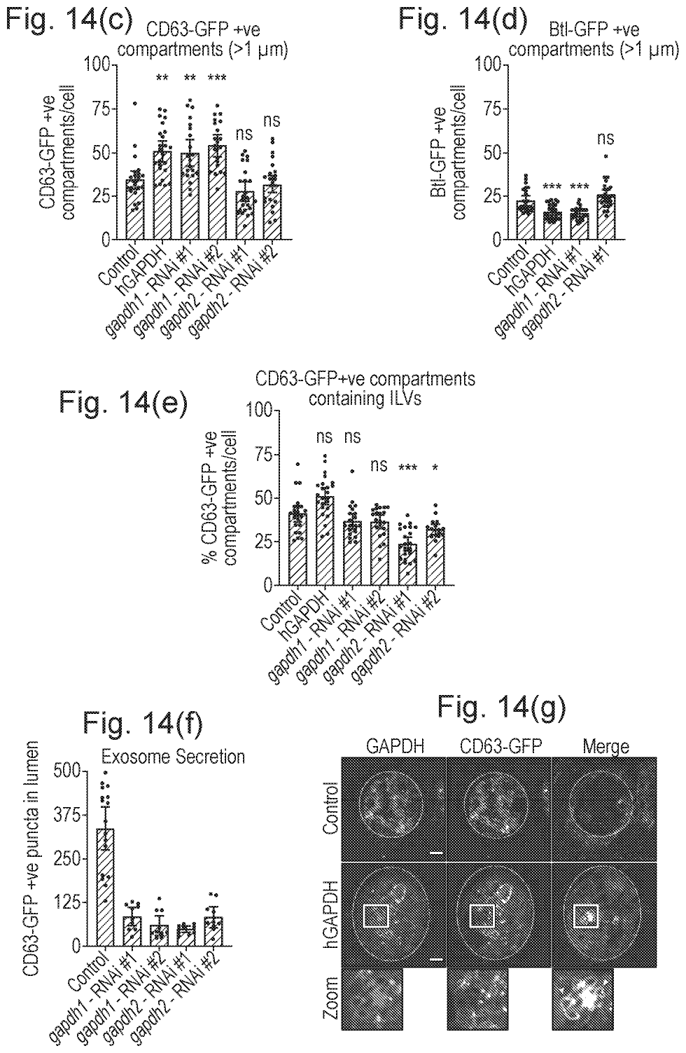

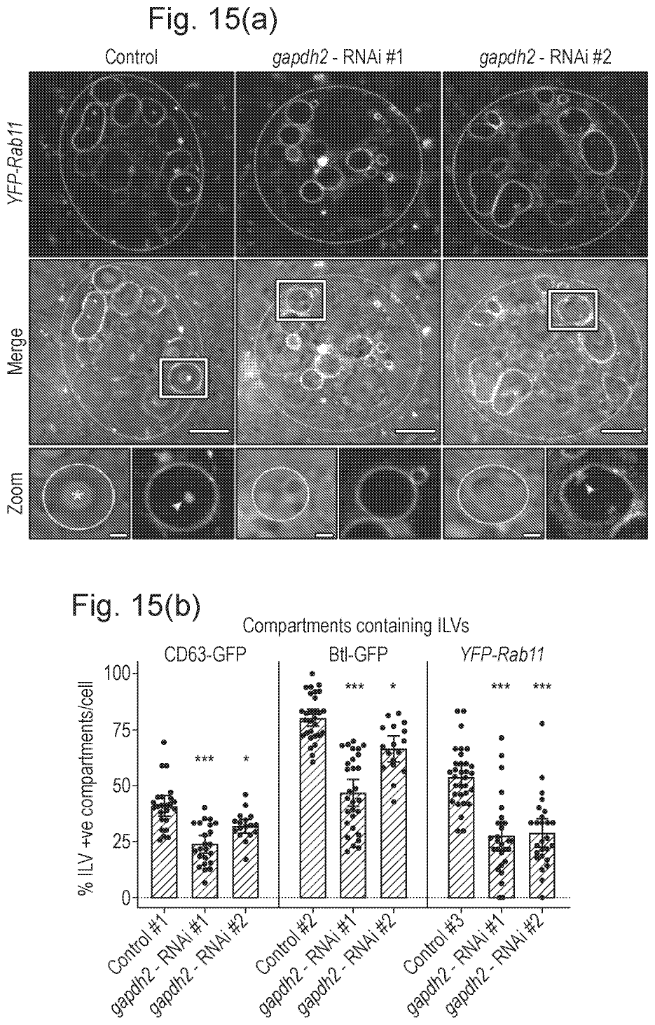

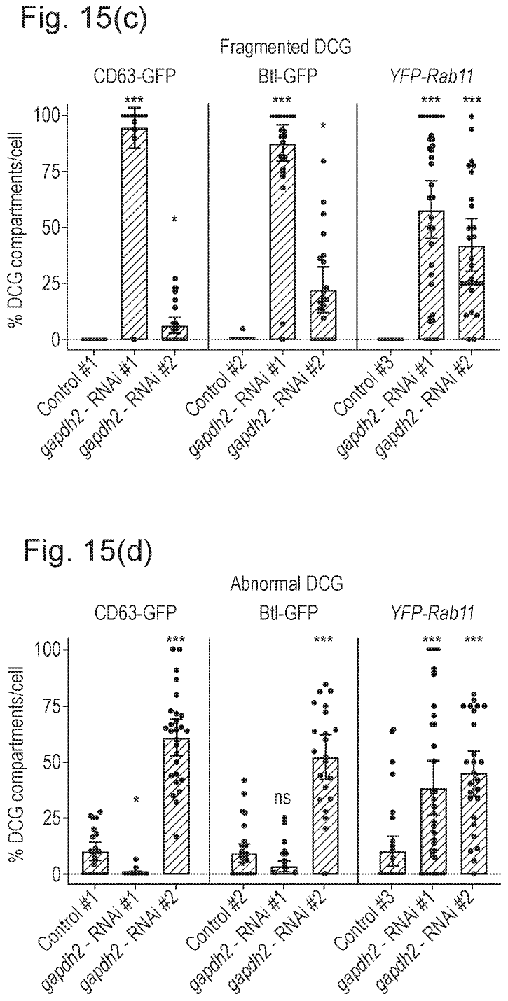

To enhance loading of lactoferrin N on EVs, the inventors increased the GAPDH concentration on EV surface. Incubation of isolated EVs with GAPDH protein resulted in extensive binding of GAPDH on the EV surface, as confirmed by immunoblotting, UV-spectrophotometry and GAPDH enzymatic activity (FIGS. 1a, 1b and 5c). Binding of GAPDH to EVs occurred in all the cell sources tested with or without the presence of serum proteins (FIGS. 5b-5d). Interestingly, incubation of EVs with GAPDH resulted in an increase in EV particle size, as determined by nanoparticle tracking analysis (FIG. 1c). Electron microscopy of HEK293T EVs after incubation with GAPDH, revealed the formation of long branched chains of EVs. (FIG. 1d). EVs derived from MSCs, HeLa cells and B16 F10 cells incubated with GAPDH protein, formed conspicuous thread like structures suggesting GAPDH induced aggregation of EVs (FIGS. 1e and 9).

### Example 8—the PS-Binding Domain of GAPDH is Responsible for Mediating Binding of GAPDH to the Outer Surface of EVs

To determine whether the PS-binding domain of GAPDH is also responsible for mediating binding of GAPDH to the outer surface of the EVs, the inventors incubated purified G58 peptide with EVs for 2 h at 4° C. and passed the complexes through gel-filtration column to separate EVs from the unbound G58 peptide. Binding of G58 peptide to EVs was assessed by western blotting, which revealed extensive binding of the peptide to the EV surface (FIG. 1f). Quantification of G58 binding on MSCs and HEK293T-derived EVs revealed approximately 1200 and 1400 G58 peptide binding sites on each EV respectively (FIG. 6a). Moreover, binding of G58 did not significantly alter the size of EVs, suggesting that the tetrameric nature of GAPDH is presumably responsible for aggregation of EVs (FIG. 1g).

### Example 9—the Physiological Role of GAPDH Binding to the EV Membrane

To assess the physiological role of GAPDH binding to the EV membrane and induction of aggregation, the inventors exploited Drosophila melanogaster as a model organism. The release of EVs from male accessory gland (AG) after modulating specifically the expression of GAPDH protein in the secondary cells (SC) of the gland was investigated. Drosophila GAPDH is highly conserved and has similar EV binding properties to human GAPDH. Moreover, exosomes are formed as intraluminal vesicles (ILVs) in highly enlarged Rab11 compartments in SCs and then secreted into the lumen of the AG, a storage site for seminal fluid. These ILVs can be selectively marked by fluorescent transmembrane markers, such as a GFP-tagged form of the FGF receptor, Breathless (Btl-GFP). In Drosophila, ILV form in clusters that surround a large dense-core granule (DCG) of aggregated protein and extend out to the limiting membrane of the Rab11 compartments. Overexpression of human GAPDH specifically in adult SCs expanded the clusters of Btl-GFP-positive ILVs, an effect that was recapitulated when ILVs were marked with a second GFP-marked transmembrane exosome marker, human CD63 (FIGS. 2a and 2b). There was also increased clustering of Btl-GFP and CD63-GFP puncta in the AG lumen. This suggests that human GAPDH promotes vesicle aggregation in vivo. To test the role of GAPDH in normal exosome biogenesis, the inventors knocked down two Drosophila GAPDH genes individually in SCs. There was no significant effect of GAPDH1 knockdown on exosome biogenesis or secretion, which may reflect the low levels of GAPDH1 reported to be expressed in adult tissues. However, GAPDH2 knockdown led to a severe disruption of dense-core granule formation in Rab11 compartments with multiple small dense-core granules formed at the limiting membrane (FIGS. 2c, 2d and 2e). Furthermore, ILVs were only located in close proximity to the limiting membrane and small DCGs; no clusters of ILVs extended from the surface of DCGs. GAPDH2 knockdown also significantly reduced exosome secretion into the AG lumen (FIGS. 2f). This phenotype could not be recapitulated by knocking down other enzymes in the glycolytic pathway, suggesting that this is not the result of general metabolic changes. This data indicates that the formation of ILVs and DCGs in Rab11 compartments of SCs may be functionally linked processes that are regulated by GAPDH2. Importantly, inhibition of GAPDH2 expression suppressed the generation of clustered ILVs and exosome secretion, suggesting that this protein normally plays an essential role in exosome biogenesis and aggregation, consistent with the clustering phenotype observed upon human GAPDH overexpression.

Defining the physiological role of GAPDH in EVs biogenesis and showing binding of the GAPDH G58 peptide to the outer surface of EVs provided a unique tool to attach therapeutic moieties to the surface of EVs. As a proof of principle study, we fused the G58 peptide to the double-stranded RNA binding domain (dsRBD) of TRBP2 (TAR RNA binding protein 2), which has high affinity towards short double stranded RNAs such as siRNA. The protein, designated as G58T, was expressed in E. coli cells and incubated with purified EVs, which resulted in highly binding of the protein to EVs (G58T EVs). Gel shift assay and spectrofluorimetric analysis revealed efficient binding of the G58T EVs with siRNA (˜500-700 siRNA binding per EVs). Moreover, bound siRNA was protected from degradation by RNase A (FIG. 7a-7c). Confocal microscopy of N2a cells treated with fluorescently labelled G58T EVs/siRNA revealed efficient uptake of the complexes by the cells (FIG. 3a). Gene silencing assays using GAPDH predesigned siRNA, however, revealed low levels of gene silencing (˜ 15%) in N2a cells that have been treated with G58T EVs/siRNA (FIG. 7e). Co-localization studies using lysotracker dyes suggested entrapment of delivered siRNA in the late-endosomes (FIG. 7d). To overcome endosomal entrapment, we investigated the attachment of different endosomolytic peptides including TAT, HA2 and the arginine rich peptide of flock house nodovirus (FHV) to the G58T protein. These peptides have been successfully used to enhance release of drugs from late-endosomes. HA2 fusion protein could not be expressed due to the toxicity of the peptide. Attachment of either two TAT peptides or FHV peptide to the G58T protein (G58T(tat)2 or G58TF, respectively) resulted highly significantly improved activity with ˜35% and 60% silencing of endogenous genes (GAPDH and HTT) genes in the cells respectively (FIGS. 3b and 7g). Further, treatment of cells with chloroquine (an endosomolytic molecule) enhanced gene silencing efficiency to around 80%, confirming entrapment of EVs in late endosomes (FIGS. 3c 7f and 7h). Taken together, these results show efficient loading and delivery of cargo such as siRNA into the cells by G58T EVs, resulting in efficient gene silencing upon attachment of endosomolytic peptides to G58T proteins.

Overexpression of human GAPDH specifically in adult SCs produced larger clusters of Btl-GFP-positive ILVs and increased clustering of Btl-GFP puncta, representing secreted exosomes, in the AG lumen (FIG. 2(a)-(d). The ILV clustering phenotype was less obvious when SCs were labelled with the CD63-GFP marker, but CD63-GFP puncta were clearly clustered in the AG lumen (FIG. 14b). Although the number of ILV-containing compartments was affected by overexpression of human GAPDH (FIGS. 14c and 14d), the proportion of these compartments that contained ILVs was not significantly altered (FIG. 2(e) and FIG. 14e. It was not possible to detect endogenous GAPDH in SCs. However, an antibody recognizing human GAPDH identified this molecule in hGAPDH-overexpressing SCs, in association with membranous structures inside late endosomal and lysosomal SC compartments, suggesting that it traffics into the endolysosomal system in SCs, as it does in human cells (FIG. 14g).

To test the role of GAPDH in normal exosome biogenesis, the two Drosophila GAPDH genes in SCs were knocked down. There was no significant effect of GAPDH1 knockdown on ILV DCG biogenesis in these cells, when using either the Btl-GFP or CD63-GFP markers, although exosome secretion was reduced (FIG. 2(a)-(d) and (g) and FIGS. 14e and f). However, GAPDH2 knockdown led to a severe disruption of DCG formation in SC compartments. There was no central dense core, but multiple small dense-cores formed near the limiting membrane (FIGS. 2(a)-(d) and (f). Btl-GFP-labelled ILVs were only located in close proximity to the limiting membrane and around the small DCGs (FIGS. 2(a)-(d) and (e); they did not cluster in non-DCG-associated chains, as seen in normal cells, demonstrating that this process is GAPDH2-dependent. A similar phenotype was observed with CD63-GFP-marked ILVs, where the proportion of compartments making ILVs was also significantly decreased (FIGS. 14b and e). In addition, GAPDH2 knockdown significantly reduced exosome secretion from these compartments into the AG lumen (FIG. 2(g) and FIG. 14f).

To check that the identity of DCG compartments had not been altered by this manipulation, we knocked down GAPDH2 in SCs expressing YFP-Rab11 from the endogenous Rab11 locus. Compartments containing defective DCGs were still labelled with YFP-Rab11 (FIG. 15). However, the proportion of these compartments making YFP-Rab11-positive ILVs was significantly decreased and those ILVs formed were closely associated with small DCGs or the limiting membrane of each compartment, confirming that GAPDH2 knockdown specifically affects ILV clustering and biogenesis. Notably, ILV and DCG phenotypes were observed with two GAPDH2-RNAi targeting different sequences, using all three exosome markers, confirming that the phenotypes did not result from an off-target effect (FIG. 14e and FIG. 15). The GAPDH2 knockdown phenotype was not recapitulated by knocking down other glycolytic enzymes, namely Phosphoglucomutase 2 (Pgm2a), Phosphoglucose isomerase (Pgi) and Phosphofructokinase (Pfk), suggesting that the observed effects on exosome biogenesis are not a consequence of general metabolic changes, but rather due to specific reduction in GAPDH2. Overall, this data indicate that the formation and clustering of ILVs and DCG biogenesis in Rab11 compartments of SCs are regulated by GAPDH2 and appear to be functionally linked processes, a result supported by a recent analysis of ESCRT function in SCs.

### Example 10—Assessing the Therapeutic Applicability of Engineered EV Animal Models

To further assess the therapeutic applicability of G58 engineered EVs animal models, the inventors investigated targeted delivery of siRNA into the mouse brain by co-expressing the RVG peptide on the surface of EVs. To determine whether binding of G58TF protein to EV surface would alter biodistribution of RVG-EVs, the inventors assessed biodistribution of systemically administered RVG-EVs in C57 BL/6 mice. After 4 h of administration, significant amount of RVG-EVs were observed in the whole brain of the mice. Binding of either G58TF or G58TF/siRNA to RVG-EVs did not change their biodistribution (FIG. 3d), although, as anticipated the majority of EVs were distributed in peripheral organs, indicating rapid clearance of EVs from the blood (FIG. 8).

To assess the silencing efficiency and therapeutic of G58TF/siRNA RVG-EVs in the brain, the inventors chose to silence, as an example, the huntingtin gene in the Huntington's disease (HD) mouse model Q140. HD is an ideal target to assess the efficiency of RNA-based drugs such as siRNA and miRNA (PMID: 12926013). Administration a total of four doses of EVs on a weekly interval resulted in 40% silencing of the HTT gene in the cortex and a significant decrease of p62 inclusion bodies in the cortical neurons of the treated animals (FIGS. 3f and 3g). p62 is an important regulatory protein of selective autophagy, and reduction in p62 aggregates in HD mice models has previously been shown to restore HD-associated phenotypes (PMID: 25305080)

### Example 11—Conjugation of G58 Peptide to Magnetic Beads for Purification of Extracellular Vesicles

The presence of ubiquitous free G58 binding sites on the surface of EVs were utilised to develop a method for isolation of EVs from different sources. As a proof of principal, G58T peptide was conjugated to magnetic beads (Dynabeads) and incubated with EVs isolated from mesenchymal stem cells (MSCs), which resulted in efficient binding of the EVs (FIG. 12).

Magnetic Dyna beads containing free COOH functional group (ThermoFisher Scientific) were activated by incubating them with N-hydroxy succinimide (NHS) and N, N′-diisopropyl-carbodiimide (DIC) in dimethylsulphoxide (DMSO) for 2 h. After the incubation, DMSO was removed by pulling the beads towards the bottom of the tube by magnetic bar. Fresh G58T protein in PBS was added to the activated beads and incubate at 4° C. for overnight. Excess of unbound G58T was removed from the beads. To assess binding of EVs, G58T-conjugated beads were incubated with fluorescently labelled EVs for 2 h at room temperature. After the incubation, beads were pulled down by magnetic force and supernatant was collected to analyse the number of EVs by NTA.

### Example 12—GAPDH Binding to EV Markers/Proteins

Incubation of EVs with recombinant dsRBD of TARBP2 did not result in EV binding, suggestive of specific binding of G58 peptide to the EV surface (FIG. 13c).

EVs derived from HEK293F cells and MSCs were analyzed by single vesicle high resolution Imaging Flow Cytometry (IFC). This method has been previously optimized extensively for detection and quantification of single fluorescently labelled EVs with an Amnis Image StreamX MkII instrument. By staining CD63-neonGFP-tagged EVs with APC-labelled anti-CD63 antibodies, it was confirmed that the Amnis Cellstream facilitates detection of single fluorescent EVs (FIG. 13d). Endogenous GAPDH was detected on both HEK293F cell and MSC derived EVs (FIG. 13e). Incubation of EVs from both cell lines with anti-GAPDH antibodies and fluorescently labelled G58 peptide resulted in detection of co-labelled EVs (FIG. 13f), thereby confirming on the single EV level that GAPDH is present on EVs derived from both cell lines and that G58 binds extensively to those EVs.

To determine whether GAPDH secretion by cells is mediated via EVs or if a non-vesicular route of GAPDH secretion also exists, a GAPDH-GFP fusion protein was expressed in HEK293T cells and isolated EVs from the cell cultured media. Analysis of GAPDH-GFP fluorescence from EVs and non-EV protein fractions reflected predominant association of GAPDH-GFP in the non-EV protein fractions, suggesting the existence of non-vesicular routes of GAPDH secretion, consistent with reports from others (FIG. 13g). In other words, GAPDH-GFP protein was expressed in the cells and after 48 h, the cell culture media of the transfected cells harvested. The media was processed to isolate EVs and non-EV fraction. Analysis of these fractions showed presence of GAPDH-GFP in both EVs and non-EV fractions, confirming that GAPDH is secreted via both EV and EV independent routes.

Thus, it has been confirmed by single cell EV fluorescent data the colocalization of GAPDH and G58 peptide with CD63GFP EVs. It has been confirmed that binding of GAPDH is specific to EVs that express EV proteins. It has also been shown that binding of GAPDH is specific and highly efficient to EVs that bear EV markers, and specifically EV proteins. The distribution of GAPDH in the cell culture media has also been demonstrated (FIG. 13g).

### Sequence Information

SEQ ID NO: 1—Sequence of the GAPDH peptide (designated as the G70 peptide based on amino acid numbering in the GAPDH protein).

NGKLVINGNPITIFQERDPSKIKWGDAGAE

SEQ ID NO: 2—Sequence of the GAPDH peptide that including extra amino acids at the N and C termini of the G70 peptide to enhance stability (designated as the G58 peptide based on amino acid numbering in the GAPDH protein; Kaneda et al; Nakagawa et al).

Underlined sequence is the sequence of the G70 peptide.

MGTVKAENGKLVINGNPITIFQERDPSKIKWGDAGAEYVVEST

Dar G H, Gopal V, Rao M. 2015. Conformation-dependent binding and tumor-targeted delivery of siRNA by a designed TRBP2: Affibody fusion protein. Nanomedicine 11(6):1455-1466.
Eguchi A, Meade B R, Chang Y C, Fredrickson C T, Willert K, Puri N, Dowdy S F. 2009. Efficient siRNA delivery into primary cells by a peptide transduction domain-dsRNA binding domain fusion protein. Nat Biotechnol 27(6):567-571.
Glaser P E, Gross R W. 1995. Rapid plasmenylethanolamine-selective fusion of membrane bilayers catalyzed by an isoform of glyceraldehyde-3-phosphate dehydrogenase: discrimination between glycolytic and fusogenic roles of individual isoforms. Biochemistry 34(38):12193-12203.
Han X, Ramanadham S, Turk J, Gross R W. 1998. Reconstitution of membrane fusion between pancreatic islet secretory granules and plasma membranes: catalysis by a protein constituent recognized by monoclonal antibodies directed against glyceraldehyde-3-phosphate dehydrogenase. Biochim Biophys Acta 1414(1-2):95-107.
Kaneda M, Takeuchi K, Inoue K, Umeda M. 1997. Localization of the phosphatidylserine-binding site of glyceraldehyde-3-phosphate dehydrogenase responsible for membrane fusion. J Biochem 122(6):1233-1240.
Lonn P, Kacsinta A D, Cui X S, Hamil A S, Kaulich M, Gogoi K, Dowdy S F. 2016. Enhancing Endosomal Escape for Intracellular Delivery of Macromolecular Biologic Therapeutics. Sci Rep 6:32301.
Malhotra H, Sheokand N, Kumar S, Chauhan A S, Kumar M, Jakhar P, Boradia V M, Raje Cl, Raje M. 2016. Exosomes: Tunable Nano Vehicles for Macromolecular Delivery of Transferrin and Lactoferrin to Specific Intracellular Compartment. J Biomed Nanotechnol 12(5):1101-1114.
Nakagawa T, Hirano Y, Inomata A, Yokota S, Miyachi K, Kaneda M, Umeda M, Furukawa K, Omata S, Horigome T. 2003. Participation of a fusogenic protein, glyceraldehyde-3-phosphate dehydrogenase, in nuclear membrane assembly. J Biol Chem 278(22):20395-20404.
Nakase I, Hirose H, Tanaka G, Tadokoro A, Kobayashi S, Takeuchi T, Futaki S. 2009. Cell-surface accumulation of flock house virus-derived peptide leads to efficient internalization via macropinocytosis. Mol Ther 17(11):1868-1876.
J. Z. Nordin, Y. Lee, P. Vader, I. Mager, H. J. Johansson, W. Heusermann, O. P. Wiklander, M. Hallbrink, Y. Seow, J. J. Bultema, J. Gilthorpe, T. Davies, P. J. Fairchild, S. Gabrielsson, N. C. Meisner-Kober, J. Lehtio, C. I. Smith, M. J. Wood, S. El Andaloussi, Ultrafiltration with size-exclusion liquid chromatography for high yield isolation of extracellular vesicles preserving intact biophysical and functional properties, Nanomedicine 11(4) (2015) 879-83.
Tisdale E J. 2001. Glyceraldehyde-3-phosphate dehydrogenase is required for vesicular transport in the early secretory pathway. J Biol Chem 276(4):2480-2486.
C. Thery, S. Amigorena, G. Raposo, A. Clayton, Isolation and characterization of exosomes from cell culture supernatants and biological fluids, Curr Protoc Cell Biol Chapter 3 (2006) Unit 3 22.

1. A composition comprising an extracellular vesicle (EV), further comprising a phosphatidylserine-binding protein and/or peptide bound to the outer surface of the EV by means of an interaction between the phosphatidylserine-binding protein and/or peptide, and a lipid and/or an EV protein on the outer surface of the EV.

2. The composition according to claim 1, wherein the lipid is the phospholipid phosphatidylserine.

3. The composition according to claim 1 or 2, wherein the phosphatidylserine-binding protein and/or peptide is selected from one or more of annexin, copine, DGK, DOC 1, DOC2, dynamin, erythrocyte protein 4.1, factor V, factor VII, factor VIII, factor IX, factor X, FGF, GAPDH, gas-6, lactadherin, MARCKS, neutral sphingomyelinase, Na/K ATPase, NO synthase, PKC, PLC, protein C, protein S, prothrombin, phosphatidylserine receptor, rabphilin, Raf-1, scavenger receptor, SK1, synaptotagmin and vinculin, and/or a phosphatidylserine-binding variant or fragment of anyone thereof.

4. The composition according to any one of the previous claims, wherein the phosphatidylserine-binding protein and/or peptide is not GAPDH, is not an annexin, is not lactadherin, and/or is not a variant or fragment of GAPDH, an annexin, and/or lactadherin.

5. The composition according to any one of the previous claims, wherein the composition is substantially devoid of vesicle aggregates; and/or the diameter of the EV is 30 to 150 nm or 150 to 1000 nm.

6. The composition according to any one of the previous claims, wherein the EV comprises 500 to 5000 molecules of the phosphatidylserine-binding protein and/or peptide bound to the outer surface of the EV.

7. The composition according to any one of claim 1, 2, 3, 5 or 6, wherein the phosphatidylserine-binding protein and/or peptide comprises:
(a) a polypeptide sequence having at least 80%, at least 90%, at least 95% or at least 100% sequence identity to SEQ ID NO: 1, optionally comprising 1-10 additional amino acids at the 5′ and/or 3′ end;
(b) a polypeptide having at least 80%, at least 90%, at least 95% or at least 100% sequence identity to SEQ ID NO: 2; and/or
(c) at least 10, at least 20 or at least 30 contiguous amino acid residues from the polypeptide sequence of SEQ ID NOs: 1 or 2.

8. The composition according to any one of the previous claims, wherein the phosphatidylserine-binding protein and/or peptide is linked to:
(a) a second protein and/or peptide; and/or
(b) a small molecule drug.

9. The composition according to claim 8, wherein the second protein, peptide and/or small molecule drug is selected from one or more of an enzyme, an antibody and/or antigen-binding variant or fragment thereof, a single chain variable fragment (scFv) and a cargo-binding protein and/or peptide.

10. The composition according to claim 9, wherein the cargo-binding protein and/or peptide is selected from one or more of an antibody and/or antigen binding variant or fragment thereof, a single chain variable fragment (scFv), a nucleic acid-binding protein and/or peptide, and a nucleic acid analogue binding protein and/or peptide.

11. The composition according to claim 10, wherein the cargo-binding protein and/or peptide is a RNA- and/or DNA-binding protein selected from one or more of TRBP2 and PKdsRBD2 and/or a RNA- and/or DNA-binding variant or fragment of anyone thereof.

12. The composition according to any one of the previous claims loaded with a cargo on the surface of the EV, wherein the cargo binds to the phosphatidylserine-binding protein and/or peptide, and/or the second protein and/or peptide.

13. The composition according to claim 12, wherein the cargo is selected from one or more of a small molecule drug, a protein, a peptide, an antibody and/or antigen binding variant or fragment thereof, a single chain variable fragment (scFv), a nucleic acid, a nucleic acid analogue, gRNA, miRNA, shRNA, siRNA, piRNA, PMO and DNA.

14. The composition according to any one of the previous claims, wherein the composition further comprises a release system, preferably wherein the release system is an organic compound-based or polypeptide-based release system such a cis-cleaving polypeptide-based release system comprising an intein.

15. The composition according to claim 14, wherein the release system comprises a linker that can be activated to release the second protein and/or cargo from the EV.

16. The composition according to any one of the previous claims, wherein the composition is co-administered with, and/or further comprises, a molecule that enhances release of the EV from endosomes.

17. The composition according to claim 16, wherein the molecule that enhances release of the EV from endosomes is a molecule that binds to protons.

18. The composition according to claim 16 or 17, wherein the molecule that enhances release of the EV from endosomes is chloroquine, or a proton binding variant thereof.

19. The composition according to any one of claim 16 to 18, wherein the molecule that enhances release of the EV from endosomes is linked to:
(a) an EV membrane-bound moiety, optionally wherein the membrane-bound moiety is cholesterol and/or a protein and/or peptide; and/or
(b) a phosphatidylserine-binding protein, optionally wherein the phosphatidylserine-binding protein is a protein and/or peptide according to any one of claim 2 or 5.

20. The composition according to any one of the previous claims, wherein the EV is an exosome.

21. The composition according to any one of the previous claims, wherein the EV further comprises at least one lipid molecule that is not phosphatidylserine.

22. The composition according to any one of the previous claims and at least one pharmaceutically acceptable excipient, for use in a method of therapy in a subject.

23. The composition according to any one of the previous claims and at least one pharmaceutically acceptable excipient, for use in a method of treating and/or preventing Alzheimer's disease, autoimmune conditions, cancer, cardiovascular disease, cystic fibrosis, Duchenne muscular dystrophy, haemophilia, Huntington's disease, lysosomal storage disease, macular degeneration, myotonic dystrophy, neuromuscular disease, Parkinson's disease, sepsis, spinal muscular atrophy or stroke.

24. A method of producing the composition according to any one of the previous claims, comprising:
(a) providing an EV expressing phosphatidylserine on the outer surface of the EV; and
(b) providing a phosphatidylserine-binding protein and/or peptide to the EV and allowing the phosphatidylserine-binding protein and/or peptide to bind to the phosphatidylserine,
thereby producing an EV composition comprising a phosphatidylserine-binding protein and/or peptide bound to the outer surface of the EV by means of an interaction between the phosphatidylserine-binding protein and/or peptide and phosphatidylserine on the outer surface of the EV.

25. The method according to claim 24, wherein the phosphatidylserine-binding protein and/or peptide is linked to a second protein and/or peptide, optionally wherein the second protein and/or peptide is selected from one or more of an enzyme, an antibody and/or antigen-binding variant or fragment thereof, a single chain variable fragment (scFv) and a cargo-binding protein

26. The method according to claim 24 or 25, further comprising providing a cargo selected from one or more of a protein, a peptide, an antibody and/or antigen binding variant or fragment thereof, a single chain variable fragment (scFv), a nucleic acid, a nucleic acid analogue, gRNA, miRNA, shRNA, siRNA, piRNA, PMO and DNA, and allowing the cargo to bind to the phosphatidylserine-binding protein and/or peptide, and/or the second protein and/or peptide.

27. The method according to any one of claim 24 to 26, wherein the EV further comprises at least one lipid molecule that is not phosphatidylserine.

28. The method according to any one of claim 24 to 27, further comprising providing a release system according to claim 14 or 15.

29. The method according to any one of claim 24 to 28, further comprising a molecule that enhances release of the EV from endosomes according to any one of claim 16 to 19.

30. A protein and/or peptide according to any one of claim 7 to 13.

31. A protein and/or peptide according to claim 30, wherein the protein and/or peptide is fused to a release system according to claim 14 or 15.

32. The use of a composition according to any one of claim 1 to 21 or a protein and/or peptide according to claim 30 or 31, for purifying an EV.

33. The in vitro or ex vivo use of a composition according to any one of claim 1 to 21 or a protein and/or peptide according to claim 30 or 31, as a research tool, a diagnostic tool, an imaging tool, biological reference material, an experimental control and/or an experimental standard.

34. The use according to claim 32 or 33, wherein the composition or protein and/or peptide is immobilised to a solid support.


SEQUENCE LISTING (2 sequences)

SEQ ID NO: 1 - protein, 30 aa, Artificial Sequence
  G70 peptide
NGKLVINGNPITIFQERDPSKIKWGDAGAE

SEQ ID NO: 2 - protein, 43 aa, Artificial Sequence
  G58 peptide
MGTVKAENGKLVINGNPITIFQERDPSKIKWGDAGAEYVVEST